(19) United States
(12) Patent Application Publication
(10) Pub. No.: US 2026/0157894 A1
(43) Pub. Date: Jun. 11, 2026

(54) VISUALLY COORDINATED DISPOSABLE ABSORBENT ARTICLES

(71) Applicant: The Procter & Gamble Company, Cincinnati, OH (US)

(72) Inventors: Amy Lynn TALLY, Cold Spring, KY (US); Astrid Annette SHEEHAN, Cincinnati, OH (US); Alizha Victoria SMITH, Wyoming, OH (US); Madison Ann WADE, Cincinnati, OH (US); Jessica Sutton EKANAYAKE, Cincinnati, OH (US)

(21) Appl. No.: 19/408,723

(22) Filed: Dec. 4, 2025

Related U.S. Application Data
(60) Provisional application No. 63/729,526, filed on Dec. 9, 2024.

(51) Int. Cl. A61F 13/84; A61F 13/49

(52) U.S. Cl. CPC A61F 13/84; A61F 13/49012; A61F 2013/8497

(57) ABSTRACT

A disposable absorbent article may include a liquid impermeable backsheet and at least two discrete elasticized members. The liquid impermeable backsheet may include a front visual signal and a back visual signal. At least one of the discrete elasticized members may have the same or substantially similar visual signal as the front visual signal and/or the back visual signal.

### CROSS REFERENCE TO RELATED APPLICATION

This application claims the benefit of U.S. Provisional Application No. 63/729,526, filed Dec. 9, 2024, the entire disclosure of which is hereby incorporated herein by reference.

### FIELD

The present disclosure relates generally to disposable absorbent articles and more specifically to disposable absorbent articles with visual signals.

### BACKGROUND

Consumers increasingly demand disposable hygienic articles that look and feel like real garments. Therefore, disposable hygienic articles should have a smooth, seamless appearance, while also providing advanced functionality. Creating this combination is a significant challenge. These articles are machine assembled from many discrete components, each with its own stretch properties, resulting in visible seams and transitions that may disrupt the desired smooth look.

Traditionally, manufacturers have used graphic designs to disguise these seams. However, as consumer preferences have shifted towards more form-fitting and contoured products, disposable articles now incorporate a variety of stretch materials and technologies within a single product. Consequently, graphics alone may be insufficient to conceal the extensive stretch features of contemporary products, which include diverse stretch forces, corrugation profiles, dimensions, and locations. Therefore, a need exists for disposable absorbent articles that are visually pleasing, particularly as modern designs incorporate diverse stretch features and consumer preferences shift towards seamless aesthetics.

The discussion of shortcomings and needs existing in the field prior to the present disclosure is in no way an admission that such shortcomings and needs were recognized by those skilled in the art prior to the present disclosure.

### SUMMARY

Various embodiments solve the above-mentioned problems and provide disposable absorbent articles comprising visually coordinated components. In contrast to employing graphic designs to conceal seams, the present disclosure enhances the visual appeal of multiple assembled parts. It has been discovered that a combination of visual cues or signals can be aesthetically pleasing and intuitively operable, even though disposable absorbent articles may still be perceived as comprising numerous assembled parts. The visual signals can be strategically arranged and designed to convey a desirable and harmonious complex functionality without compromising softness and comfort sensations.

These and other features, aspects, and advantages of various embodiments will become better understood with reference to the following description, figures, and claims.

### BRIEF DESCRIPTION OF THE DRAWINGS

The above-mentioned and other features and advantages of the present disclosure, and the manner of attaining them, will become more apparent and the disclosure itself will be better understood by reference to the following description of example forms of the disclosure taken in conjunction with the accompanying drawings, wherein:

FIG. 1 is a plan view of an example absorbent article in the form of a taped diaper, garment-facing surface facing the viewer, in a flat laid-out state;

FIG. 2 is a plan view of the example absorbent article of FIG. 1, wearer-facing surface facing the viewer, in a flat laid-out state;

FIG. 3 is a front perspective view of the absorbent article of FIGS. 1 and 2 in a fastened position;

FIG. 4 is a front perspective view of an absorbent article in the form of a pant;

FIG. 5 is a rear perspective view of the absorbent article of FIG. 4;

FIG. 6 is a plan view of the absorbent article of FIG. 4, laid flat, with a garment-facing surface facing the viewer;

FIG. 7 is a cross-sectional view of the absorbent article taken about line 7-7 of FIG. 6;

FIG. 8 is a cross-sectional view of the absorbent article taken about line 8-8 of FIG. 6;

FIG. 9 is a plan view of an example absorbent core or an absorbent article;

FIG. 10 is a cross-sectional view, taken about line 10-10, of the absorbent core of FIG. 9;

FIG. 11 is a cross-sectional view, taken about line 11-11, of the absorbent core of FIG. 9;

FIG. 12 is a plan view of an example absorbent article of the present disclosure that is a sanitary napkin;

FIG. 13 is a plan view of an example absorbent article in the form of a taped diaper, garment-facing surface facing the viewer, in a flat laid-out state;

FIG. 14 is a front view of a diamond pattern ultrasonically bonded elastic laminate in a partially stretched state;

FIG. 15 is a perspective view of a diamond pattern ultrasonically bonded elastic laminate in a relaxed state;

FIG. 16 is a perspective view of an absorbent article comprising a front waist region in a relaxed state;

FIG. 17 is a perspective view of an absorbent article comprising a back waist region in a relaxed state;

FIG. 18 is a perspective view of an absorbent article, laid flat, with a garment-facing surface facing the viewer to show an exterior front waist region comprising an ultrasonically bonded elastic laminate and an exterior back waist region comprising an ultrasonically bonded elastic laminate;

FIG. 19 is a perspective view of an absorbent article, laid flat, with a wearer-facing surface facing the viewer to show an interior front waist region comprising an ultrasonically bonded elastic laminate and an interior back waist region comprising an ultrasonically bonded elastic laminate;

FIG. 20 is a plan view of several example absorbent articles showing the front and back waist regions of the back sheets;

FIG. 21 is a perspective view of back ears attached to a back sheet fully within a back sheet visual signal area;

FIGS. 22A-FIG. 22L are schematic illustrations of some exemplary ultrasonic bond patterns;

FIG. 23 is a chart plotting extension vs force curves for example back ears;

FIG. 24A is a front view of an absorbent article comprising visually coordinated components;

FIG. 24B is a back view of an absorbent article comprising visually coordinated components;

FIG. 24C is a side view of an absorbent article comprising visually coordinated components;

FIG. 24D is a front-side perspective view of an absorbent article comprising visually coordinated components;

FIG. 24E is a back-side perspective view of an absorbent article comprising visually coordinated components;

FIG. 25 A-F are cross-sectional views of various stretch laminates; and

FIG. 26 is a diagram of a diamond shape.

It should be understood that the various embodiments are not limited to the examples illustrated in the figures.

### DETAILED DESCRIPTION

### Introduction and Definitions

This disclosure is written to describe the invention to a person having ordinary skill in the art, who will understand that this disclosure is not limited to the specific examples or embodiments described. The examples and embodiments are single instances of the invention which will make a much larger scope apparent to the person having ordinary skill in the art. Unless defined otherwise, all technical and scientific terms used herein have the same meaning as commonly understood by the person having ordinary skill in the art. It is also to be understood that the terminology used herein is for the purpose of describing examples and embodiments only, and is not intended to be limiting, since the scope of the present disclosure will be limited only by the appended claims.

All the features disclosed in this specification (including any accompanying claims, abstract, and drawings) may be replaced by alternative features serving the same, equivalent, or similar purpose, unless expressly stated otherwise. Thus, unless expressly stated otherwise, each feature disclosed is one example only of a generic series of equivalent or similar features. The examples and embodiments described herein are for illustrative purposes only and that various modifications or changes in light thereof will be suggested to the person having ordinary skill in the art and are to be included within the spirit and purview of this application. Many variations and modifications may be made to the embodiments of the disclosure without departing substantially from the spirit and principles of the disclosure. All such modifications and variations are intended to be included herein within the scope of this disclosure. For example, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

All numeric values are herein assumed to be modified by the term “about,” whether or not explicitly indicated. The term “about” generally refers to a range of numbers that one of skill in the art would consider equivalent to the recited value (for example, having the same function or result). In many instances, the term “about” may include numbers that are rounded to the nearest significant figure.

In everyday usage, indefinite articles (like “a” or “an”) precede countable nouns and noncountable nouns almost never take indefinite articles. It must be noted, therefore, that, as used in this specification and in the claims that follow, the singular forms “a,” “an,” and “the” include plural referents unless the context clearly dictates otherwise. Thus, for example, reference to “a support” includes a plurality of supports. Particularly when a single countable noun is listed as an element in a claim, this specification will generally use a phrase such as “a single.” For example, “a single support.”

Unless otherwise specified, all percentages indicating the amount of a component in a composition represent a percent by weight of the component based on the total weight of the composition. The term “mol percent” or “mole percent” generally refers to the percentage that the moles of a particular component are of the total moles that are in a mixture. The sum of the mole fractions for each component in a solution is equal to 1.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

In this specification and in the claims that follow, reference will be made to a number of terms that shall be defined to have the following meanings unless a contrary intention is apparent.

“Absorbent article” refers to devices that absorb and contain liquid, and more specifically, refers to devices that are placed against or in proximity to the body of the wearer to absorb and to contain various exudates discharged from the body.

“Align” or “aligned” or “aligning” means to place or to arrange in a straight line. Aligning edges of substrates, therefore, means arranging the substrates so that the edges in question extend along approximately the same line. It is to be appreciated that aligning edges of substrates can be accomplished in a variety of ways, including placing the substrates one on top of the other or side by side.

“Facing relationship” refers to a relative positioning of materials, such as substrates, in which a surface of one material is oriented toward a surface of another material. For example, when two substrates are stacked on top of each other, they are in a facing relationship. The term does not require or exclude the presence of intervening objects, materials, or layers.

“Machine direction” (MD) refers to the direction of material flow through a process. In addition, relative placement and movement of material can be described as flowing in the machine direction through a process from upstream in the process to downstream in the process.

“Cross direction” (CD) refers to a direction that is generally perpendicular to the machine direction.

Various non-limiting forms of the present disclosure will now be described to provide an overall understanding of the principles of the structure, function, manufacture, and use of the visually coordinated disposable absorbent articles disclosed herein. One or more examples of these non-limiting forms are illustrated in the accompanying drawings. Those of ordinary skill in the art will understand that the visually coordinated disposable absorbent articles described herein and illustrated in the accompanying drawings are non-limiting example forms and that the scope of the various non-limiting forms of the present disclosure are defined solely by the claims. The features illustrated or described in connection with one non-limiting form may be combined with the features of other non-limiting forms. Such modifications and variations are intended to be included within the scope of the present disclosure.

### General Description of an Absorbent Article

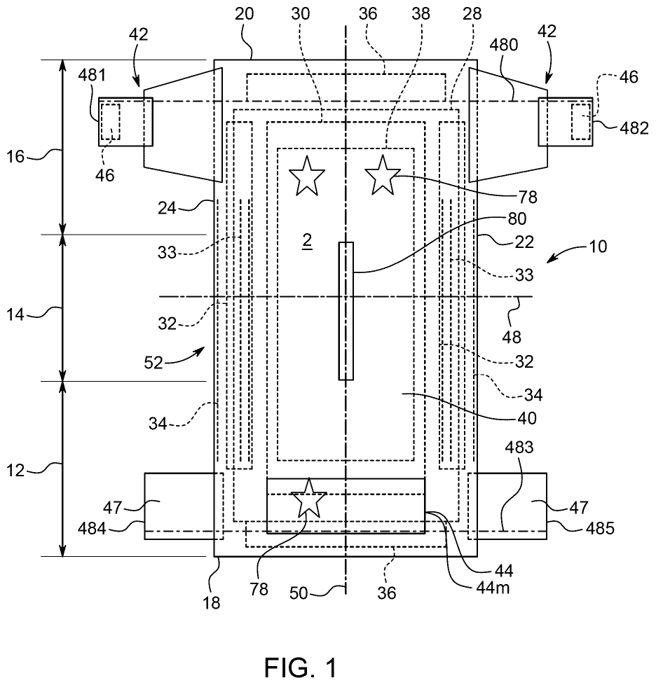

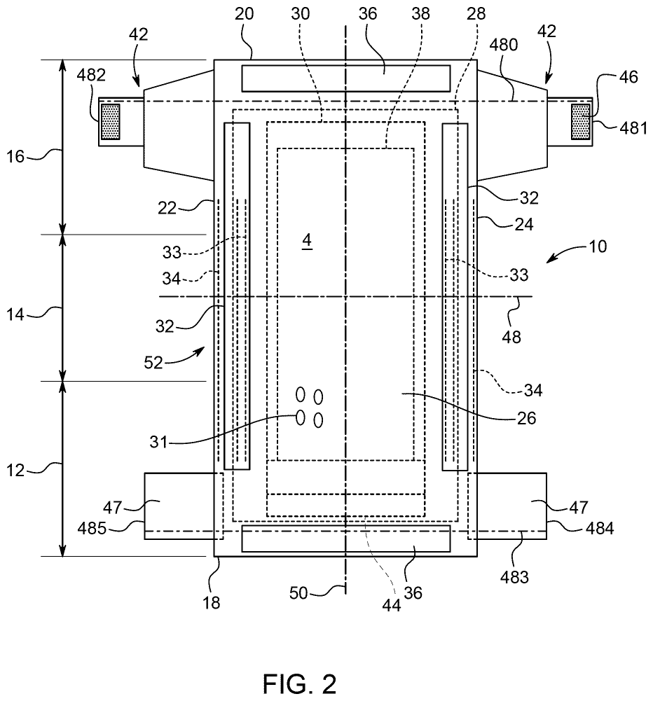

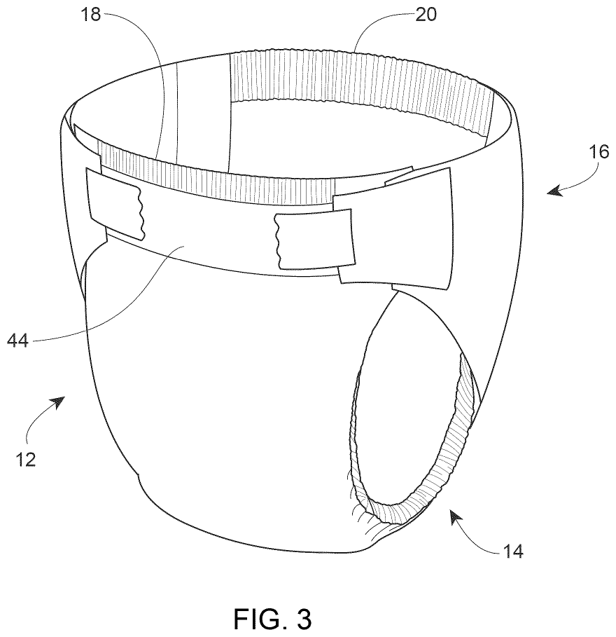

An example absorbent article 10 according to the present disclosure, shown in the form of a taped diaper, is represented in FIGS. 1-3. FIG. 1 is a plan view of the example absorbent article 10, garment-facing surface 2 facing the viewer in a flat, laid-out state (e.g., no elastic contraction). FIG. 2 is a plan view of the example absorbent article 10 of FIG. 1, wearer-facing surface 4 facing the viewer in a flat, laid-out state. FIG. 3 is a front perspective view of the absorbent article 10 of FIGS. 1 and 2 in a fastened configuration. The absorbent article 10 of FIGS. 1-3 is shown for illustration purposes only as the present disclosure may be used for making a wide variety of diapers, including adult incontinence products, pants, or other absorbent articles, such as sanitary napkins and absorbent pads, for example.

The absorbent article 10 may comprise a front waist region 12, a crotch region 14, and a back waist region 16. The crotch region 14 may extend intermediate the front waist region 12 and the back waist region 16. The front waist region 12, the crotch region 14, and the back waist region 16 may each be ⅓ of the length of the absorbent article 10. The absorbent article 10 may comprise a front end edge 18, a back end edge 20 opposite to the front end edge 18, and longitudinally extending, transversely opposed side edges 22 and 24 defined by the chassis 52.

The absorbent article 10 may comprise a liquid permeable topsheet 26, a liquid impermeable backsheet 28, and an absorbent core 30 positioned at least partially intermediate the topsheet 26 and the backsheet 28. The absorbent article 10 may also comprise one or more pairs of barrier leg cuffs 32 with or without elastics 33, one or more pairs of leg elastics 34, one or more elastic waistbands 36, and/or one or more acquisition materials 38. The acquisition material or materials 38 may be positioned intermediate the topsheet 26 and the absorbent core 30. An outer cover material 40, such as a nonwoven material, may cover a garment-facing side of the backsheet 28. The absorbent article 10 may comprise back ears 42 in the back waist region 16. The back ears 42 may comprise fasteners 46 and may extend from the back waist region 16 of the absorbent article 10 and attach (using the fasteners 46) to the landing zone area 44 or landing zone material 44m on a garment-facing portion of the front waist region 12 of the absorbent article 10. The absorbent article 10 may also have front ears 47 in the front waist region 12. The absorbent article 10 may have a central lateral (or transverse) axis 48 and a central longitudinal axis 50. The central lateral axis 48 extends perpendicular to the central longitudinal axis 50.

In other instances, the absorbent article may be in the form of a pant having permanent or refastenable side seams. Suitable refastenable seams are disclosed in U.S. Pat. Appl. Pub. No. 2014/0005020 and U.S. Pat. No. 9,421,137.

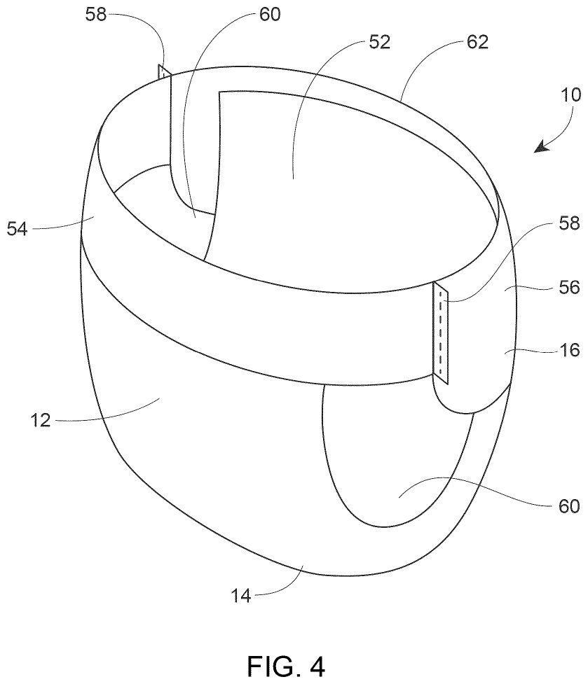

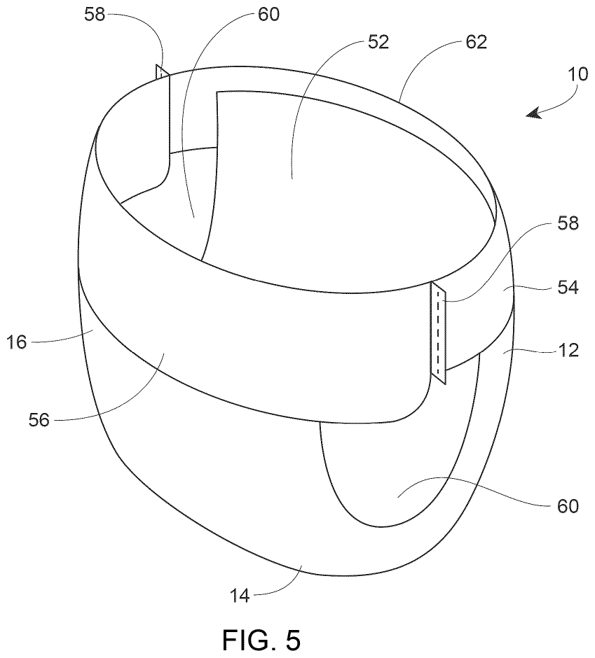

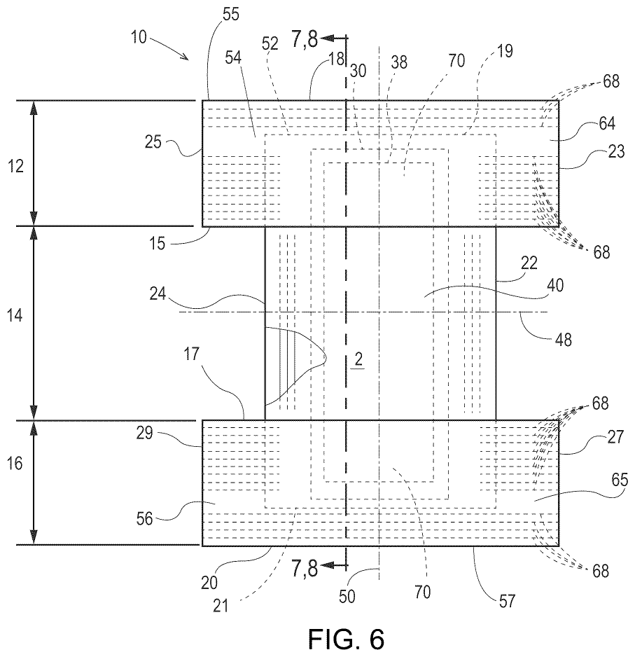

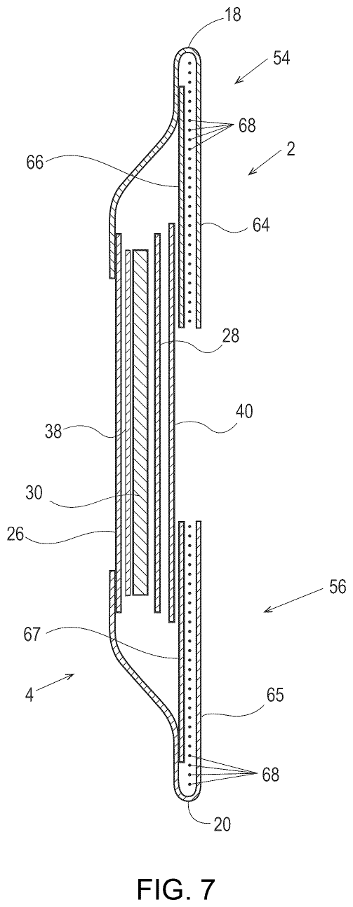

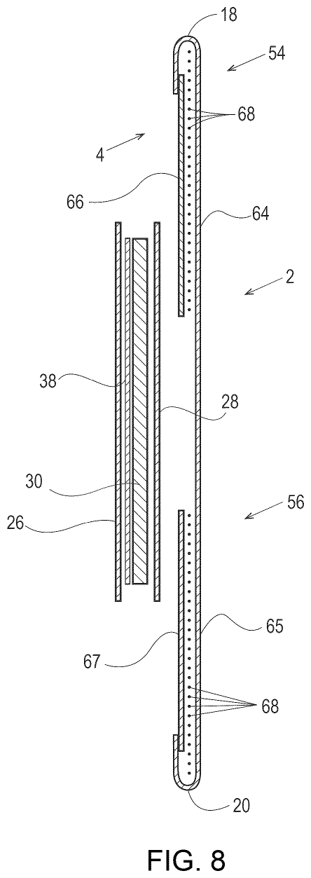

Referring to FIGS. 4-8, an example absorbent article 10 in the form of a pant is illustrated. FIG. 4 is a front perspective view of the absorbent article 10. FIG. 5 is a rear perspective view of the absorbent article 10. FIG. 6 is a plan view of the absorbent article 10, laid flat, with the garment-facing surface facing the viewer. Elements of FIG. 4-8 having the same reference number as described above with respect to FIGS. 1-3 may be the same element (e.g., absorbent core 30). FIG. 7 is an example cross-sectional view of the absorbent article taken about line 7-7 of FIG. 6. FIG. 8 is an example cross-sectional view of the absorbent article taken about line 8-8 of FIG. 6. FIGS. 7 and 8 illustrate example forms of front and back belts 54, 56. The absorbent article 10 may have a front waist region 12, a crotch region 14, and a back waist region 16. Each of the regions 12, 14, and 16 may be ⅓ of the length of the absorbent article 10. The absorbent article 10 may have a chassis 52 (sometimes referred to as a central chassis or central panel) comprising a topsheet 26, a backsheet 28, and an absorbent core 30 disposed at least partially intermediate the topsheet 26 and the backsheet 28, and an optional acquisition material 38, similar to that as described above with respect to FIGS. 1-3. The absorbent article 10 may comprise a front belt 54 in the front waist region 12 and a back belt 56 in the back waist region 16. The chassis 52 may be joined to a wearer-facing surface 4 of the front and back belts 54, 56 or to a garment-facing surface 2 of the belts 54, 56. Side edges 23 and 25 of the front belt 54 may be joined to side edges 27 and 29, respectively, of the back belt 56 to form two side seams 58. The side seams 58 may be any suitable seams known to those of skill in the art, such as butt seams or overlap seams, for example. When the side seams 58 are permanently formed or refastenably closed, the absorbent article 10 in the form of a pant has two leg openings 60 and a waist opening circumference 62. The side seams 58 may be permanently joined using adhesives or bonds, for example, or may be refastenably closed using hook and loop fasteners, for example.

### Belts

Referring to FIGS. 7 and 8, the front and back belts 54 and 56 may comprise front and back inner belt layers 66 and 67 and front and back outer belt layers 64 and 65 having an elastomeric material (e.g., strands 68 or a film (which may be apertured)) disposed at least partially therebetween. The elastic elements 68 or the film may be relaxed (including being cut) to reduce elastic strain over the absorbent core 30 or, may alternatively, run continuously across the absorbent core 30. The elastics elements 68 may have uniform or variable spacing therebetween in any portion of the belts. The elastic elements 68 may also be pre-strained the same amount or different amounts. The front and/or back belts 54 and 56 may have one or more elastic element free zones 70 where the chassis 52 overlaps the belts 54, 56. In other instances, at least some of the elastic elements 68 may extend continuously across the chassis 52.

The front and back inner belt layers 66, 67 and the front and back outer belt layers 64, 65 may be joined using adhesives, heat bonds, pressure bonds or thermoplastic bonds. Various suitable belt layer configurations can be found in U.S. Pat. No. 9,072,632.

Front and back belt end edges 55 and 57 may extend longitudinally beyond the front and back chassis end edges 19 and 21 (as shown in FIG. 6) or they may be co-terminus. The front and back belt side edges 23, 25, 27, and 29 may extend laterally beyond the chassis side edges 22 and 24. The front and back belts 54 and 56 may be continuous (e.g., having at least one layer that is continuous) from belt side edge to belt side edge (e.g., the transverse distances from 23 to 25 and from 27 to 29). Alternatively, the front and back belts 54 and 56 may be discontinuous from belt side edge to belt side edge (e.g., the transverse distances from 23 to 25 and 27 to 29), such that they are discrete.

As disclosed in U.S. Pat. No. 7,901,393, the longitudinal length (along the central longitudinal axis 50) of the back belt 56 may be greater than the longitudinal length of the front belt 54, and this may be particularly useful for increased buttocks coverage when the back belt 56 has a greater longitudinal length versus the front belt 54 adjacent to or immediately adjacent to the side seams 58.

The front outer belt layer 64 and the back outer belt layer 65 may be separated from each other, such that the layers are discrete or, alternatively, these layers may be continuous, such that a layer runs continuously from the front belt end edge 55 to the back belt end edge 57. This may also be true for the front and back inner belt layers 66 and 67—that is, they may also be longitudinally discrete or continuous. Further, the front and back outer belt layers 64 and 65 may be longitudinally continuous while the front and back inner belt layers 66 and 67 are longitudinally discrete, such that a gap is formed between them - a gap between the front and back inner and outer belt layers 64, 65, 66, and 67 is shown in FIG. 7 and a gap between the front and back inner belt layers 66 and 67 is shown in FIG. 8. In some configurations shown in FIGS. 7 and 8, portions of the outer belt layers 64, 65 may be folded over onto the inner belt layers, respectively. In addition, as shown in FIG. 7, portions of the outer belt layers 64, 65 may also be folded over onto the chassis 52.

The front and back belts 54 and 56 may include slits, holes, and/or perforations providing increased breathability, softness, and a garment-like texture. Underwear-like appearance can be enhanced by substantially aligning the waist and leg edges at the side seams 58 (see FIGS. 4 and 5).

The front and back belts 54 and 56 may comprise graphics (see e.g., 78 of FIG. 1). The graphics may extend substantially around the entire circumference of the absorbent article 10 and may be disposed across side seams 58 and/or across proximal front and back belt edges 15 and 17; or, alternatively, adjacent to the seams 58 and/or proximal front and back belt edges 15 and 17 in the manner described in U.S. Pat. No. 9,498,389 to create a more underwear-like article. The graphics may also be discontinuous. In some configurations, the graphics may be disposed on at least a portion of the belts, the inner belt layers 66, 67, and/or outer belt layers 64,65 comprising elastic elements 68 and, in such configurations, the graphics may complement and be coordinated visually with any bond patterns in elastic regions or in other regions of the absorbent article 10. In some configurations, the graphics may be disposed on, or visible on, the garment facing surface 2 of the belts 54,56, the wearer-facing surface 4 of the belts 54, 56, or a combination of both.

Alternatively, instead of attaching belts 54 and 56 to the chassis 52 to form a pant, discrete side panels may be attached to side edges of the chassis 22 and 24. Suitable forms of pants comprising discrete side panels are disclosed in U.S. Pat. Nos. 6,645,190; 8,747,379; 8,372,052; 8,361,048; 6,761,711; 6,817,994; 8,007,485; 7,862,550; 6,969,377; 7,497,851; 6,849,067; 6,893,426; 6,953,452; 6,840,928; 8,579,876; 7,682,349; 7,156,833; and 7,201,744.

### Topsheet

The topsheet 26 is the part of the absorbent article 10 that is in contact with the wearer's skin. The topsheet 26 may be joined to portions of the backsheet 28, the absorbent core 30, the barrier leg cuffs 32, and/or any other layers as is known to those of ordinary skill in the art. The topsheet 26 may be compliant, soft-feeling, and non-irritating to the wearer's skin. Further, at least a portion of, or all of, the topsheet may be liquid permeable, permitting liquid bodily exudates to readily penetrate through its thickness. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. The topsheet may have one or more layers. The topsheet may be apertured (FIG. 2, element 31), may have any suitable three-dimensional features, and/or may have a plurality of embossments (e.g., a bond pattern). The topsheet may comprise a variable basis weight nonwoven material, such as those described in U.S. Pat. Appl. No. 2017/0191198. The topsheet may be apertured by overbonding a material and then rupturing the overbonds through ring rolling, such as disclosed in U.S. Pat. No. 5,628,097, to Benson et al., issued on May 13, 1997, and disclosed in U.S. Pat. Appl. Publication No. US 2016/0136014 to Arora et al. Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet. The topsheet may comprise a one or more layer of hydroentangled material with or without apertures. The topsheet may comprise a variable basis weight nonwoven material. At least a portion of the topsheet 26, for example in the front waist region 12 and/or back waist region 16, may comprise apertures, or arrays of apertures, having a similar pattern to, or visually coordinated with, one or more bond patterns, 3D patterns, 2D patterns, or printed patterns on any other surfaces or materials on the garment-facing surface 2 or wearer-facing surface 4 of the absorbent article 10 in the front waist region 12 and/or back waist region 16 of the absorbent article 10.

### Backsheet

The backsheet 28 is generally that portion of the absorbent article 10 positioned proximate to the garment-facing surface of the absorbent core 30. The backsheet 28 may be joined to portions of the topsheet 26, the outer cover material 40, the absorbent core 30, and/or any other layers of the absorbent article by any attachment methods known to those of skill in the art. The backsheet 28 prevents, or at least inhibits, the bodily exudates absorbed and contained in the absorbent core 10 from soiling articles such as bedsheets, undergarments, and/or clothing. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials, such as films, which permit vapors to escape from the absorbent article, while still preventing, or at least inhibiting, bodily exudates from passing through the backsheet. The backsheet 28 may be coterminous with the outer cover material 40. At least a portion of the backsheet, 28, for example in the front waist region 12 and/or back waist region 16, may be printed or mechanically modified to have a texture or visible pattern having a similar pattern to, or visually coordinated with, one or more bond patterns, 3D patterns, apertures, 2D patterns, or printed patterns on any other surfaces or materials on the garment-facing surface and/or wearer-facing surface 4 of the absorbent article 10 in the front waist region 12 and/or back waist region 16 of the absorbent article 10.

### Outer Cover Material

The outer cover material (sometimes referred to as a backsheet nonwoven) 40 may comprise one or more nonwoven materials joined to the backsheet 28 and that covers the backsheet 28. The outer cover material 40 forms at least a portion of the garment-facing surface 2 of the absorbent article 10 and effectively “covers” the backsheet 28 so that film is not present on the garment-facing surface 2. The outer cover material 40 may comprise a bond pattern, apertures, and/or three-dimensional features. The outer cover material 40 may be a hydroentangled nonwoven material or a variable basis weight nonwoven material. Such a material may have one or more layers. At least a portion of the outer cover material 40, for example in the front waist region 12 and/or back waist region 16, may be printed or mechanically modified to have a texture or visible pattern having a similar pattern to, or visually coordinated with, one or more bond patterns, 3D patterns, apertures, 2D patterns, or printed patterns on any other surfaces or materials on the garment-facing surface 2 and/or wearer-facing surface 4 of the absorbent article 10 in the front waist region 12 and/or back waist region 16 of the absorbent article 10.

### Absorbent Core

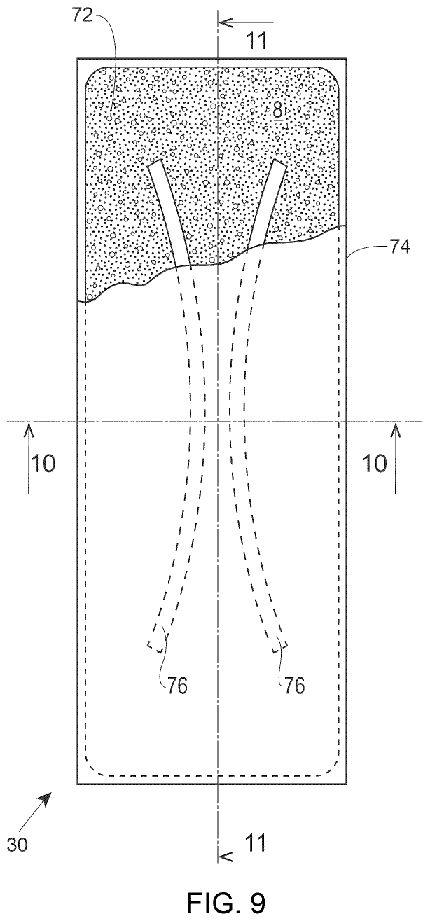

As used herein, the term “absorbent core” refers to a component of the absorbent article 10 for absorbing and containing liquid such as urine received by the absorbent article. The absorbent core thus typically has a high absorbent capacity. An example absorbent core 30 is schematically shown in FIGS. 9-11. The absorbent core comprises an absorbent material 72. The absorbent typically comprises a core wrap 74, which may also be referred to as a core bag 74, that encloses or sandwiches the absorbent material.

The core wrap may be a single material that is folded and attached to itself, or it may comprise a separate top layer and bottom layer that may be bonded, adhesively joined, or otherwise joined together. The top and bottom layers of the core wrap may be the same or different. The absorbent material typically comprises superabsorbent particles which are optionally mixed with cellulose fibers. As used herein, “absorbent core” does not include any acquisition-distribution systems, topsheet, or backsheet of the absorbent article.

The example absorbent core 30 shown in isolation in FIGS. 9-11 is in the dry state (before use). The absorbent core may typically have a generally rectangular shape as defined by its longitudinal edges and transversal front edge and back edge or may have other shapes.

Absorbent material 72 may be deposited as an absorbent layer having a generally rectangular outline, as represented in FIG. 9. A wide variety of absorbent cores may also be used. The absorbent material 72 layer may also have a non-rectangular perimeter (“shaped” core), in particular, the absorbent material 72 may define a tapering along its width towards the central region of the core (or “dog-bone” shape). In this way, the absorbent material deposition area may have a relatively narrow width in an area of the core intended to be placed in the crotch region of the absorbent article. This may provide for example better wearing comfort. Other shapes can also be used such as a “T” or “Y” or “hourglass” for the area of the absorbent material.

The absorbent material 72 may be any conventional absorbent material known in the art. For example, the absorbent material may comprise a blend of cellulose fibers and superabsorbent particles (“SAP”), typically with the percentage of SAP ranging from about 50% to about 75% by weight of the absorbent material. The absorbent material may also be free of cellulose fibers, as is known in so-called airfelt-free cores, where the absorbent material consists, or consists essentially, of SAP. The absorbent material may also be a high internal phase emulsion foam. High loft nonwoven materials may be present in the core bags, or proximate but outside the core bags. The absorbent material may comprise one or more layers of a coform material. The coform material may comprise a mixture of fibers and an absorbent material. The fibers may comprise staple fibers such as synthetic fibers or absorbent fibers. The synthetic fibers may comprise polypropylene fibers, polyethylene fibers, and/or bicomponent fibers. The absorbent fibers may comprise pulp, lyocell, and/or viscose. The fibers may also comprise thermoplastic filaments (scattered or interconnected networks). The absorbent material may comprise a super absorbent polymeric material in fiber or particle form. The one or more layers of coform material may comprise virgin or recycled materials. The staple fibers may be present in the coform material in an amount of from about 5 wt. % to about 50 wt. %. The absorbent material may be present in the nonwoven web in an amount of from about 50 wt. % to about 95 wt. %. The staple fibers may have an average length of from about 5 mm to about 50 mm. The staple fibers and absorbent fibers may be thermally bonded or hydraulically entangled to form the nonwoven web (as disclosed in WO2023/022979).

“Superabsorbent polymer” or “SAP” refers herein to absorbent materials, typically cross-linked polymeric materials, that can absorb at least 10 times their weight of an aqueous 0.9% saline solution as measured using the Centrifuge Retention Capacity (CRC) test (EDANA method WSP 241.2.R3 (12)). The SAP may in particular have a CRC value of at least 20 g/g, in particular of from about 20 g/g to about 40 g/g. “Superabsorbent polymer particles”, as used herein, refers to a superabsorbent polymer material which is in particulate form so as to be flowable in the dry state.

Various absorbent core designs comprising high amounts of SAP have been proposed in the past, see for example in U.S. Pat. No. 5,599,335 (Goldman), EP1,447,066 (Busam), WO95/11652 (Tanzer), U.S. Pat. Appl. Pub. No. 2008/0312622A1 (Hundorf), WO2012/052172 (Van Malderen). In particular, the SAP printing technology as disclosed in U.S. Pat. No. 7,838,722 (Blessing), U.S. Pat. No. 9,072,634 and U.S. Pat. No. 8,206,533 (both to Hundorf et al.) may be used. The present disclosure however is not limited to a particular type of absorbent core. The absorbent core may also comprise one or more glues such as an auxiliary glue applied between the internal surface of one (or both) of the core wrap layers and the absorbent material to reduce leakage of SAP outside the core wrap. A micro-fibrous adhesive net may also be used in air-felt free cores as described in the above Hundorf references. These glues are not represented in the Figures for simplicity. Other core constructions comprising a high loft nonwoven substrate, such as a carded nonwoven layer, having a porous structure into which SAP particles have been deposited, may also be used in present disclosure.

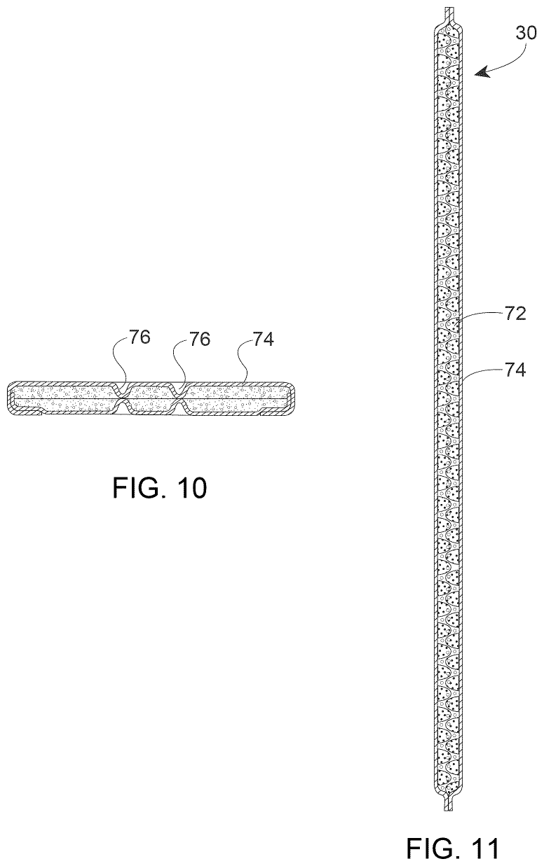

The absorbent material may be deposited as a continuous layer within the core wrap. The absorbent material may also be present discontinuously, for example, as individual pockets or stripes of absorbent material enclosed within the core wrap and separated from each other by material-free junction areas. A continuous layer of absorbent material, in particular of SAP, may also be obtained by combining two absorbent layers having matching discontinuous absorbent material application pattern, wherein the resulting layer is substantially continuously distributed across the absorbent particulate polymer material area, as illustrated in FIGS. 10-11. As for example taught in U.S. Pat. Appl. Pub. No. 2008/0312622A1 (Hundorf), each absorbent material layer may thus comprise a pattern having absorbent material land areas and absorbent material-free junction areas, wherein the absorbent material land areas of the first layer correspond substantially to the absorbent material-free junction areas of the second layer and vice versa.

The basis weight (amount deposited per unit of surface) of the absorbent material may also be varied to create a profiled distribution of absorbent material, in particular in the longitudinal direction to provide more absorbency towards the center and the middle of the core, but also in the transversal direction, or both directions of the core. The absorbent core may also comprise one or more longitudinally (or otherwise) extending channels 76, which are areas of the absorbent layer substantially free of absorbent material within the absorbent material layer. The top side of the core wrap may be advantageously bonded to the bottom side of the core by adhesive, mechanical or ultra-sonic bonding through these material-free areas. Example disclosures of such channels in an airfelt-free core can be found in U.S. Pat. No. 9,789,011. One or more channels may also be formed in absorbent cores comprising a mix of cellulose fibers and SAP particles. These channels may embody any suitable shapes and any suitable number of channels may be provided. In other instances, the absorbent core may be embossed to create the impression of channels, with absorbent material remaining within the channels. The absorbent core in FIGS. 9-11 is merely an example absorbent core. Many other absorbent cores with or without channels are also within the scope of the present disclosure.

### Barrier Leg Cuffs/Leg Elastics

Referring to FIGS. 1 and 2, for example, the absorbent article 10 may comprise one or more pairs of barrier leg cuffs 32 and one or more pairs of leg elastics 34. The barrier leg cuffs 32 may be positioned laterally inboard of leg elastics 34. Each barrier leg cuff 32 may be formed by a piece of material which is bonded to the absorbent article 10 so it can extend upwards from a wearer-facing surface 4 of the absorbent article 10 and provide improved containment of body exudates approximately at the junction of the torso and legs of the wearer. The barrier leg cuffs 32 are delimited by a proximal edge joined directly or indirectly to the topsheet and/or the backsheet and a free terminal edge, which is intended to contact and form a seal with the wearer's skin. The barrier leg cuffs 32 may extend at least partially between the front end edge 18 and the back end edge 20 of the absorbent article 10 on opposite sides of the central longitudinal axis 50 and may be at least present in the crotch region 14. The barrier leg cuffs 32 may each comprise one or more elastics 33 (e.g., elastic strands or strips) near or at the free terminal edge. These elastics 33 cause the barrier leg cuffs 32 to help form a seal around the legs and torso of a wearer. The leg elastics 34 extend at least partially between the front end edge 18 and the back end edge 20. The leg elastics 34 essentially cause portions of the absorbent article 10 proximate to the chassis side edges 22, 24 to help form a seal around the legs of the wearer. The leg elastics 34 may extend at least within the crotch region 14. The barrier leg cuffs 32 may comprise one or more layers of nonwoven material. The nonwoven material may be hydroentangled.

### Elastic Waistband

Referring to FIGS. 1 and 2, the absorbent article 10 may comprise one or more elastic waistbands 36. The elastic waistbands 36 may be positioned on the garment-facing surface 2 or the wearer-facing surface 4. Alternatively, the elastic waistbands may be positioned intermediate the topsheet and the backsheet. As an example, a first elastic waistband 36 may be present in the front waist region 12 near the front end edge 18 and a second elastic waistband 36 may be present in the back waist region 16 near the back end edge 20. The elastic waistbands 36 may aid in sealing the absorbent article 10 around a waist of a wearer and at least inhibiting bodily exudates from escaping the absorbent article 10 through the waist opening circumference. In some instances, an elastic waistband may fully surround the waist opening circumference of an absorbent article. The elastic waistband may comprise an elastic film joined to the topsheet and a nonwoven material covering the elastic film. In other instances, the elastic waistband may comprise an elastic film sandwiched between two nonwoven materials. The elastic film may be ultrasonically bonded, or otherwise bonded or attached, to the one or more nonwoven materials. The one or more nonwoven materials may be hydroentangled. The elastic film and/or the nonwoven materials may be preactivated (i.e., activated prior to being joined together) or the formed elastic film/nonwoven laminate may be activated post laminate formation. At least a portion of the elastic waistband 36, or a component thereof, may be printed or mechanically modified to have a texture or visible pattern having a similar pattern to, or visually coordinated with, one or more bond patterns, 3D patterns, apertures, 2D patterns, or printed patterns on any other surfaces or materials on the garment-facing surface 2 and/or wearer-facing surface 4 of the absorbent article 10 in the front waist region 12 and/or back waist region 16 of the absorbent article 10.

### Acquisition Materials

Referring to FIGS. 1, 2, 7, and 8, one or more acquisition materials 38 may be present at least partially intermediate the topsheet 26 and the absorbent core 30. The acquisition materials 38 are typically hydrophilic materials that provide significant wicking of bodily exudates. These materials may dewater the topsheet 26 and quickly move bodily exudates into the absorbent core 30. The acquisition materials 38 may comprise one or more nonwoven materials, foams, formed films, apertured formed films, cellulosic materials, cross-linked cellulosic materials, air laid cellulosic nonwoven materials, spunlace materials, or combinations thereof, for example. The acquisition material may be rectangular or may be shaped, such as hourglass shaped. Typically, an acquisition material 38 may have a width and length that are smaller than the width and length of the topsheet 26. The acquisition material may be a secondary topsheet in the feminine pad context. The acquisition materials may have one or more channels as described above with reference to the absorbent core 30 (including the embossed version). The channels in the acquisition material may align or not align with channels in the absorbent core 30. In an example, a first acquisition material may comprise a nonwoven material and as second acquisition material may comprise a cross-linked cellulosic material.

### Landing Zone

Referring to FIGS. 1 and 2, the absorbent article 10 may have a landing zone area 44 that is formed in a portion of the garment-facing surface 2 of the outer cover material 40. The landing zone area 44 may be in the back waist region 16 if the absorbent article 10 fastens from front to back or may be in the front waist region 12 if the absorbent article 10 fastens back to front. In some instances, the landing zone 44 may be or may comprise one or more discrete nonwoven materials that are attached to a portion of the outer cover material 40 in the front waist region 12 or the back waist region 16 depending upon whether the absorbent article fastens in the front or the back. In essence, the landing zone 44 is configured to receive the fasteners 46 and may comprise, for example, a plurality of loops configured to be engaged with, a plurality of hooks on the fasteners 46, or vice versa.

At least a portion of the landing zone may be printed or mechanically modified to have a texture or visible pattern having a similar pattern to, or visually coordinated with, one or more bond patterns, 3D patterns, apertures, 2D patterns, or printed patterns on any other surfaces or materials on the garment-facing surface 2 and/or wearer-facing surface 4 of the absorbent article in the front waist region 12 and/or the back waist region 16 of the absorbent article 10.

### Wetness Indicator/Graphics

Referring to FIG. 1, the absorbent articles 10 of the present disclosure may comprise graphics 78 and/or wetness indicators 80 that are visible from the garment-facing surface 2. The graphics 78 may be printed on the landing zone 44, the backsheet 28, and/or at other locations. The wetness indicators 80 are typically applied to the absorbent core facing side of the backsheet 28, so that they can be contacted by bodily exudates within the absorbent core 30. In some instances, the wetness indicators 80 may form portions of the graphics 78. For example, a wetness indicator may appear or disappear and create/remove a character within some graphics. In other instances, the wetness indicators 80 may coordinate (e.g., same design, same pattern, same color) or not coordinate with the graphics 78.

### Front and Back Ears

Referring to FIGS. 1 and 2, as referenced above, the absorbent article 10 may have front and/or back ears 47, 42 in a taped diaper context. Only one set of ears may be required in most taped diapers. The single set of ears may comprise fasteners 46 configured to engage the landing zone or landing zone area 44. If two sets of ears are provided, in most instances, only one set of the ears may have fasteners 46, with the other set being free of fasteners. Any ears with fasteners may be configured to engage an opposing ear or the landing zone or landing zone area 44. One set of ears may comprise primary fasteners and the other set of ears may comprise secondary fasteners. The ears, or portions thereof, may be elastic or may have elastic panels, for example, to form stretchable ears. In an example, an elastic film or elastic strands may be positioned intermediate a first nonwoven material and a second nonwoven material. One or more of the first and second nonwoven materials may be hydroentangled. The elastic film may or may not be apertured. The ears may be shaped. The ears may be integral (e.g., extension of the outer cover material 40, the backsheet 28, and/or the topsheet 26) or may be discrete components attached to a chassis 52 of the absorbent article on a wearer-facing surface 4, on the garment-facing surface 2, or intermediate the two surfaces 4, 2. The back ears may comprise an elastic film sandwiched between two nonwoven materials forming a laminate. The elastic film and/or the nonwoven materials may be pre-activated (i.e., activated prior to being joined together) and the laminate may be joined by ultrasonic bonds. Such laminates may be used in the construction of belts, elastic waistbands, and stretchable ears and are disclosed in U.S. Pat. No. 10,485,713 and in published U.S. Pat. App. No. 2024/0252361A1. In other instances, the elastic film may not be preactivated and instead the laminate may be activated after the laminate is formed. Such laminates may also be ultrasonically bonded.

Various elements, including elastomeric ears, waistbands, and/or belts, of the absorbent articles described herein may comprise a stretch laminate. Such laminates may comprise an elastomeric layer that provides extensibility to the laminate and one or more outer layers that are less stretchable but suitable for providing durability and desirable tactile properties. In this way, the laminate permits a component of an absorbent article to closely and comfortably contact the wearer while providing desirable exterior qualities.

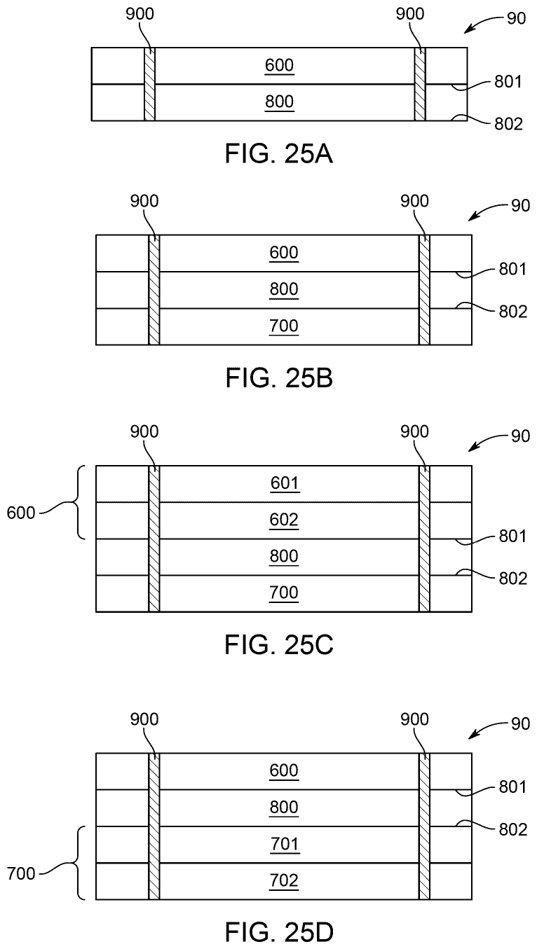

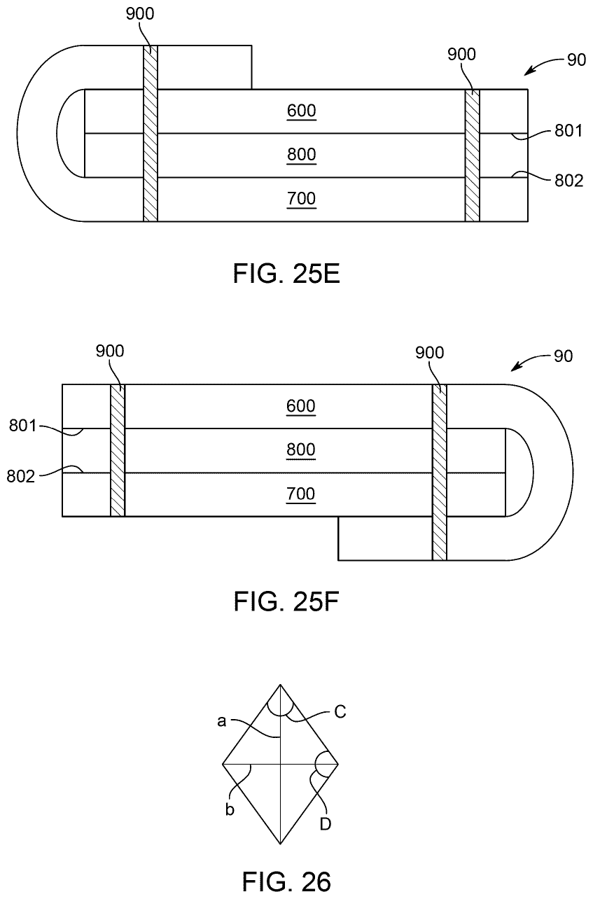

As shown in FIG. 25A, a stretch laminate 90 may comprise an elastomeric film layer 800 joined under tension (e.g., in an elongated state) via one or more ultrasonic bonds 900 to a first cover layer 600. The cover layers discussed herein may comprise one or more nonwoven materials or may comprise other types of materials. It is also to be appreciated that the layers of the stretch laminate may be bonded using a combination of mechanical bonds, such as thermal bonds, pressure bonds, and ultrasonic bonds. The elastomeric film layer 800 may comprise one or more skins, such as a first skin 801 providing first surface and a second skin 802 providing second surface. As shown in FIG. 25B, a stretch laminate 90 may comprise a first cover layer 600 and a second cover layer 700 with an elastomeric film layer 800 sandwiched therebetween in a facing relationship to both the first cover layer 600 and the second cover layer 700. All three layers may be joined via one or more ultrasonic bonds 900. As shown in FIGS. 25C and 25D, all or a portion of the first cover layer 600 may comprise one or more layers, such as a first layer 601 and a second layer 602, which may have the same composition or different compositions or the same or different properties, such as fiber type or thickness. Similarly, all or a portion of the second cover layer 700 may comprise one or more layers, such as a first layer 701 and a second layer 702, which may have the same composition or different compositions or the same or different properties, such as fiber type or thickness. As shown in FIGS. 25E and 25F, a portion of the first cover layer 600 or the second cover layer 700 may be folded over to provide a multi-layered structure on all or a portion of the opposite side of stretch laminate 90.

Elastomeric film layer 800 of stretch laminate 90 may comprise a single layer or multiple layers of one or more materials that are elastically extensible.

At least a portion of either or both the front ears 47 and/or back ears 42 and/or a component thereof, may be printed or mechanically modified to have a texture or visible pattern having a similar pattern to, or visually coordinated with, one or more bond patterns, 3D patterns, apertures, or 2D or printed patterns on any other surfaces or materials on the garment-facing surface 2 and/or wearer-facing surface 4 of the absorbent article 10 in the front waist region 12 and/or the back waist region 16 of the absorbent article 10.

### Masking Layer

One or more masking layers or materials may be provided in the absorbent articles 10. A masking layer may be a layer that provides a cushiony feel when the absorbent article is touched from the garment-facing surface 2 or the wearer-facing surface 4. The masking layer may “mask” a grainy feel potentially caused by the absorbent material 72, such as superabsorbent polymers. The masking layer may “mask” bodily exudates from being visible when viewing the wearer-facing surface 4 or the garment-facing surface 2 of the absorbent article 10. The masking layer may have a basis weight in the range of about 15 gsm to about 50 gsm or about 15 gsm to about 40 gsm. The masking layer may comprise one or more nonwoven materials (e.g., a hydroentangled nonwoven material), foams, pulp layers, and/or other suitable materials. The masking layer may be the outer cover material 40. The masking layer may be the layer forming the garment-facing side or the wearer-facing side of the core bag 74. The masking layer may be a separate material positioned intermediate the garment-facing side of the core bag 74 and the liquid impermeable backsheet 28.

### Packages

The absorbent articles of the present disclosure may be placed into packages. The packages may comprise polymeric films, paper, and/or other materials. Graphics and/or indicia relating to properties of the absorbent articles may be formed on, printed on, positioned on, and/or placed on outer portions of the packages. Each package may comprise a plurality of absorbent articles. The absorbent articles may be packed under compression so as to reduce the size of the packages, while still providing an adequate amount of absorbent articles per package. By packaging the absorbent articles under compression, caregivers can easily handle and store the packages, while also providing distribution savings to manufacturers owing to the size of the packages. The packages may comprise polymeric films comprising recycled material, such as about 20% to about 100%, about 30% to about 90%, about 30% to about 80%, about 40% to about 60%, or about 50% recycled material. The recycled material may comprise post-industrial recycled material (PIR) and/or post-consumer recycled material (PCR). In some instances, the polymeric films used for the packages may comprise two outer layers and one or more inner layers. The one or more inner layers may comprise the recycled material or may comprise more recycled material than the outer layers. The recycled material may comprise recycled polyethylene. The recycled material may comprise recycled polyethylene PIR from trim from the packaging operation.

The package material may comprise paper, paper based material, paper with one or more barrier layers, or a paper/film laminate. The package material may be in the range of about 50 gsm to about 100 gsm or about 70 gsm to about 90 gsm and the one or more barrier layers may be in the range of about 3 gsm to about 15 gsm. The paper based package material with or without one or more barrier layers may exhibit a machine direction tensile strength of at least 5.0 kN/m, a machine direction stretch of at least 3 percent, a cross-machine direction tensile strength of at least 3 kN/m, and a cross-direction stretch at break of at least 4 percent, each as determined via ISO 1924-3.

The paper based package material or paper based package material comprising a barrier layer or film may be recyclable or recyclable in normal paper recycling operations. The recyclability extent of the paper based package may be determined via recyclable percentage. The paper based package of the present disclosure may exhibit recyclable percentages of 70 percent or greater, 80 percent or greater, or 90 percent or greater. The paper based package of the present disclosure may have a recyclable percentage of between 70 percent to about 99.9 percent, between about 80 percent to about 99.9 percent, or between about 90 percent to about 99.9 percent. In one example, the package material of the present disclosure may exhibit a recyclable percentage of from about 95 percent to about 99.9 percent, from about 97 percent to about 99.9 percent, or from about 98 percent to about 99.9 percent. The recyclable percentage of the paper based package may be determined via test PTS-RH:021/97 (Draft October 2019) under category II, as performed by Papiertechnische Stiftung located at Pirnaer Strasse 37, 01809 Heidenau, Germany. In another instance, the paper based packages of the present disclosure may exhibit an overall “pass” test outcome as determined by PTS-RH:021/97 (Draft October 2019) under category II method. Any of the paper based packages may have opening features, such as lines of perforation, and may also have handles.

### Arrays

“Array” means an offering of packages of different, but related, products. For instance, an array may comprise disposable absorbent articles of different article constructions (e.g., different elastomeric materials [compositionally and/or structurally] in the side panels, back ears, side flaps and/or belts flaps, different graphic elements, different product structures, fasteners, waistbands, or lack thereof). The packages may have the same brand and/or sub-brand and/or the same trademark registration and/or having been manufactured by or for a common manufacturer and the packages may be available at a common point of sale (e.g. oriented in proximity to each other in a given area of a retail store). An array is often marketed as a line-up of products normally having like packaging elements (e.g., packaging material type, film, paper, dominant color, design theme, etc.) that convey to consumers that the different individual packages are part of a larger line-up. Arrays often have the same brand, for example, “Huggies,” and same sub-brand, for example, “Pull-Ups.” A different product in the array may have the same brand “Huggies” and the sub-brand “Little Movers.” The differences between the “Pull-Ups” product of the array and the “Little Movers” product in the array may include product form, application style, different fastening designs or other structural elements intended to address the differences in physiological or psychological development. Furthermore, in this example, the packaging is distinctly different in that “Pull-Ups” is packaged in a predominately blue or pink film bag and “Little Movers” is packaged in a predominately red film bag.

Further an array may be formed by different products having different product forms manufactured by the same manufacturer, for example, “Kimberly-Clark”, and bearing a common trademark registration. For example, one product may have the brand name “Huggies,” and sub-brand, for example, “Pull-Ups.” A different product in the array may have a brand/sub-brand “Good Nites,” both of which are registered trademarks of The Kimberly-Clark Corporation and/or are manufactured by Kimberly-Clark. Arrays also often have the same trademarks, including trademarks of the brand, sub-brand, and/or features and/or benefits across the line-up.

More broadly speaking, part of an array may be located in a physical store, while another part of the array is offered on-line. For instance, an array may include “Pampers Swaddlers,” “Pampers Cruisers,” and “Pampers Splashers,” each manufactured by or on behalf of The Procter & Gamble Company. “Pampers Swaddlers” and “Pampers Cruisers” may be available physically in stores on shelf displays in near proximity to one another, while “Pampers Splashers” may only be available on-line, but each could be considered part of the array. In another example of an array, “Pampers Pure taped diapers,” “Pampers Pure training underwear” and “Pampers Aqua Pure wipes” are each a different product type/form and have the same brand name “Pampers” and sub-brand name portion “Pure” to indicate that they are fragrance-free and/or hypoallergenic versions of the product. Without regard to where the “Pure” products are located, they may be considered part of the same array. In this example, all three “Pure” products are manufactured by or on behalf of The Procter & Gamble Company. For example, each of the aforementioned “Pure” products may be offered on-line, each may be located physically on the same shelf display, or a combination of one or two of the “Pure” products may be physically located at a store, while the other one or two “Pure” products are offered on-line. So, for example, an array may include “Pampers Pure taped diapers” and “Pampers Pure training underwear” on a shelf display at a store, while “Pampers Aqua Pure wipes” is at an alternate location (e.g., a warehouse) and digitally represented on-line. Alternatively, each of the “Pure” products may be offered on-line. A product of an array (including each product of an array) may be offered be offered on-line, as well as in stores. On-line offerings may include digital representation(s) of the physical package(s) and/or digital representation(s) of the physical product(s) within the package(s).

### Sanitary Napkin

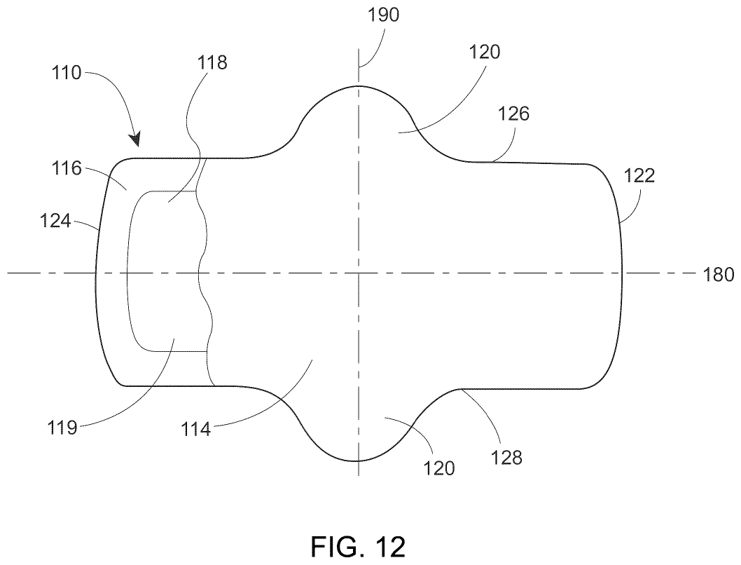

Referring to FIG. 12, an absorbent article of the present disclosure may be a sanitary napkin 110. The sanitary napkin 110 may comprise a liquid permeable topsheet 114, a liquid impermeable, or substantially liquid impermeable, backsheet 116, and an absorbent core 118. The liquid impermeable backsheet 116 may or may not be vapor permeable. The absorbent core 118 may have any or all of the features described herein with respect to the absorbent core 30 and, in some forms, may have a secondary topsheet 119 (STS) instead of the acquisition materials disclosed above. The STS 119 may comprise one or more channels, as described above (including the embossed version). In some forms, channels in the STS 119 may be aligned with channels in the absorbent core 118. The sanitary napkin 110 may also comprise wings 120 extending outwardly with respect to a longitudinal axis 180 of the sanitary napkin 110. The sanitary napkin 110 may also comprise a lateral axis 190. The wings 120 may be joined to the topsheet 114, the backsheet 116, and/or the absorbent core 118. The sanitary napkin 110 may also comprise a front edge 122, a back edge 124 longitudinally opposing the front edge 122, a first side edge 126, and a second side edge 128 longitudinally opposing the first side edge 126. The longitudinal axis 180 may extend from a midpoint of the front edge 122 to a midpoint of the back edge 124. The lateral axis 190 may extend from a midpoint of the first side edge 126 to a midpoint of the second side edge 128. The sanitary napkin 110 may also be provided with additional features commonly found in sanitary napkins as is known in the art.

### Fasteners

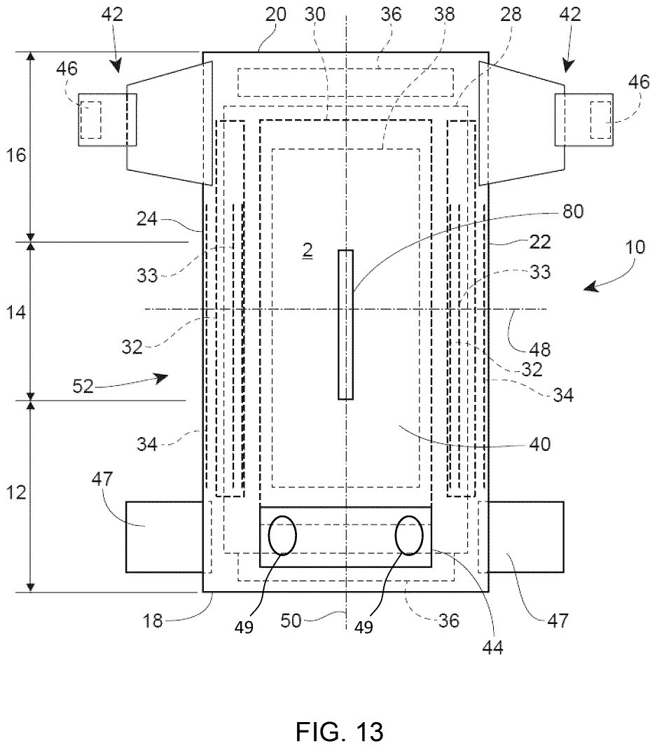

Referring to FIG. 13, the back ears 42 may comprise fasteners 46. The fasteners 46 may be configured to cooperate with the landing zone area 44 or landing zone material 44m on the garment-facing surface of the front waist region 12 of the absorbent article 10. Additionally, the absorbent article may comprise one or more secondary fasteners 49. The secondary fasteners 49 may be disposed on the outer cover material 40 or a component of the absorbent article, such as the landing zone 44 as illustrated in FIG. 13. The secondary fasteners may be a separate material joined to the absorbent article. The secondary fasteners may be integrally formed from a material of the absorbent article. For example, the secondary fasteners may comprise one or more hooks or protrusions formed from the material of the outer cover material 40, the landing zone material 44m, or a film. The hooks or protrusions may be formed using the process described in U.S. Pat. No. 8,784,722 to Rocha et al. The secondary fasteners may be configured to cooperate with a portion of the back ears. For example, a secondary fastener comprising hooks or protrusions are configured to engage the nonwoven material of the back ear. It is to be appreciated that the secondary fasteners may be any mechanical fastener that is configured to cooperate with a component of the absorbent article.

At least a portion of one or more of the fasteners 46 and/or secondary fasteners 49 may be printed or mechanically modified to have a texture or visible pattern having a similar pattern to, or visually coordinated with, one or more bond patterns, 3D patterns, apertures, 2D patterns, or printed patterns on any other surfaces or materials on the garment-facing surface 2 and/or wearer-facing surface 4 of the absorbent article 10 in the front waist region 12 and/or rear waist region 16 of the absorbent article 10, including, for example the back ears 42 and/or the landing zone 44.

### Visual Coordination

Various configurations may provide a disposable absorbent article comprising visually coordinated components. Such configurations may enhance the visual appeal of the multiple assembled components that make up the disposable absorbent articles. Although a disposable absorbent article may still be perceived as comprising numerous assembled parts, coordinated visual signals on certain components of the absorbent article may render the absorbent article aesthetically pleasing to consumers. The visual signals may be strategically arranged and designed to convey a desirable and harmonious complex functionality without compromising softness and comfort sensations.

Referring to FIGS. 1-3 and 14-21, various configurations relate to a disposable absorbent article 10 that may comprise a liquid permeable topsheet 26, a liquid impermeable backsheet 28, an absorbent core 30 positioned at least partially intermediate the topsheet 26 and the backsheet 28, and at least two discrete elasticized members, each attached to a landing zone 44 on the absorbent article 10 on a wearer facing side 4 of the absorbent article 10 or a garment-facing side 2 of the absorbent article 10. The landing zone of one of the discrete elasticized members may be free from overlap with the landing zone of the other discrete elasticized member. The at least two discrete elasticized members may comprise a front waistband 36f, a back waistband 36b, a pair of front ears 47, or a pair of back ears 42. The liquid permeable backsheet 28 may comprise a front waist region 12 and a back waist region 16 separated from each other in a longitudinal direction LD, such as along longitudinal axis 180. The liquid permeable backsheet 28 may comprise a front visual signal 202 in the front waist region 12 and a back visual signal 204 in the back waist region 16. At least one of the discrete elasticized members 36f, 36b, 42, 47 may have the same or substantially similar visual signal 206 as the front visual signal 202 and the back visual signal 204. The visual signals 202, 204, 206 may comprise color, printed repeat pattern, mechanically imparted repeat pattern or combinations thereof. The color may comprise grey, mint green, yellow, teal, pink, red, orange, or white. The printed repeat pattern or mechanically imparted repeat pattern may comprise a parallelogram, a linear array of shapes, or an offset array of shapes. The printed repeat patterns or the mechanically-imparted repeat patterns may remain substantially the same when the absorbent article is relaxed or stretched.

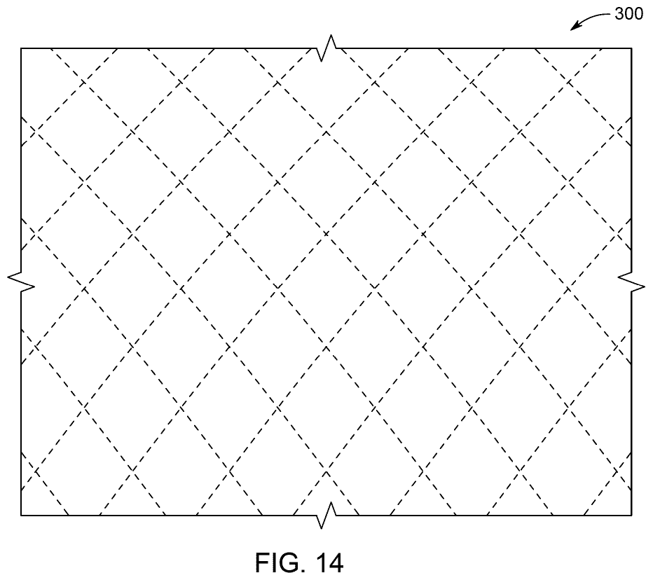

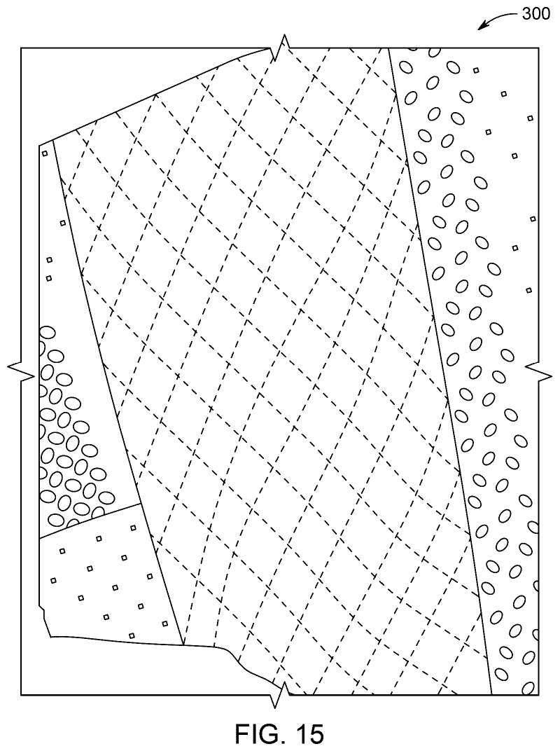

FIG. 14 is a front view of a diamond pattern ultrasonically bonded elastic laminate 300 in a partially stretched state. FIG. 15 is a perspective view of a diamond pattern ultrasonically bonded elastic laminate 300 in a relaxed state. In some configurations, at least two of the discrete elasticized members 36f, 36b, 42, 47 may comprise an ultrasonically bonded laminate 300. Of course, it is to be appreciated that all of the discrete elasticized members 36f, 36b, 42, 47 may comprise an ultrasonically bonded laminate 300. The laminate 300 may comprise a preactivated film, skins with wrinkles and furrows or visible lines of preactivation, as described herein. Any or all of the discrete elasticized members 36f, 36b, 42, 47 may have a minimum extension at load force of 400 g of from about 1 mm to about 20 mm, about 2 mm to about 15 mm, about 3 mm to about 10 mm, about 4 mm to about 9 mm, about 5 mm to about 8 mm, about 6 mm to about 7 mm, or about 5 mm. Any or all of the discrete elasticized members 36f, 36b, 42, 47 may have a minimum extension at load force of 1000 g of from about 10 mm to about 40 mm, about 15 mm to about 30 mm, about 16 mm to about 25 mm, about 17 mm to about 24 mm, about 18 mm to about 23 mm, about 19 to about 22 mm, about 20 mm to about 21 mm, or about 22 mm.

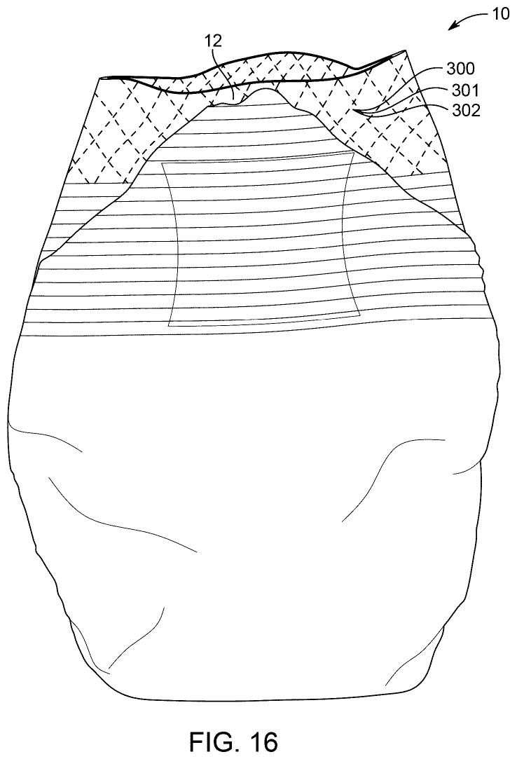

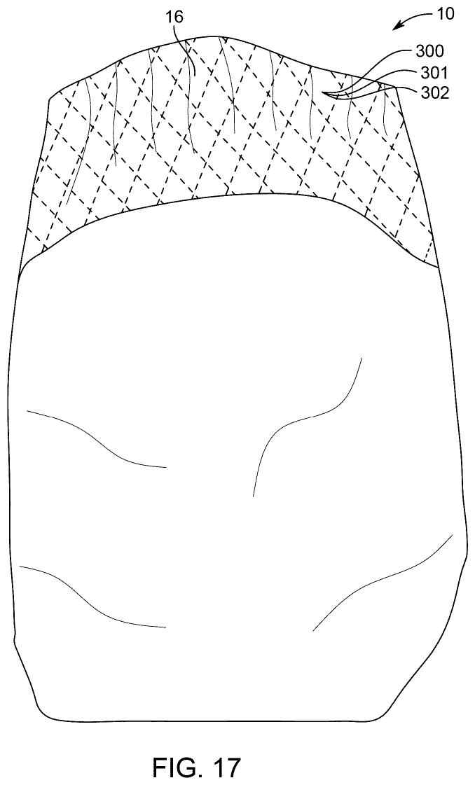

FIG. 16 is a perspective view of an absorbent article 10 comprising a front waist region 12 in a relaxed state. The front waist region 12 may comprise a laminate 301. The laminate 301 may comprise a diamond patterned ultrasonically bonded elastic laminate 300. In some configurations, the laminate 301 may comprise a printed diamond pattern 302 that may correspond with a diamond patterned ultrasonically bonded elastic laminate 300, which may be associated with the front waist region 12, and/or with another component of the absorbent article 10. In some configurations, the laminate 301 may not comprise a printed diamond pattern 302. FIG. 17 is a perspective view of an absorbent article 10 comprising a back waist region 16 in a relaxed state. The back waist region 16 may comprise a laminate 301. The laminate 301 may comprise a diamond patterned ultrasonically bonded elastic laminate 300. In some configurations, the laminate 301 may comprise a printed diamond pattern 302 that corresponds with a diamond patterned ultrasonically bonded elastic laminate 300, which may be associated with the back waist region 16, and/or with another component of the absorbent article 10. It is to be appreciated that the absorbent article 10 may comprise a taped diaper or any of the absorbent articles described herein. In some configurations, the laminate 301 may not comprise a printed diamond pattern 302.

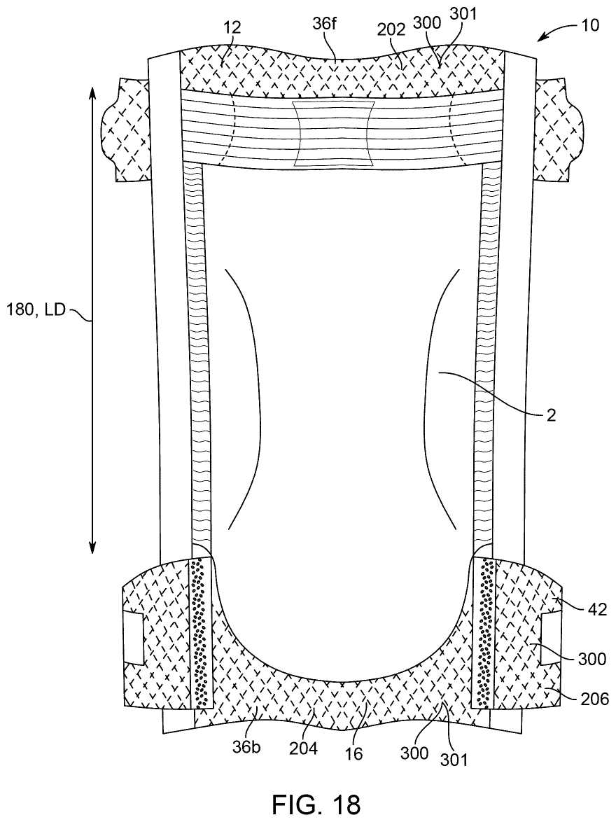

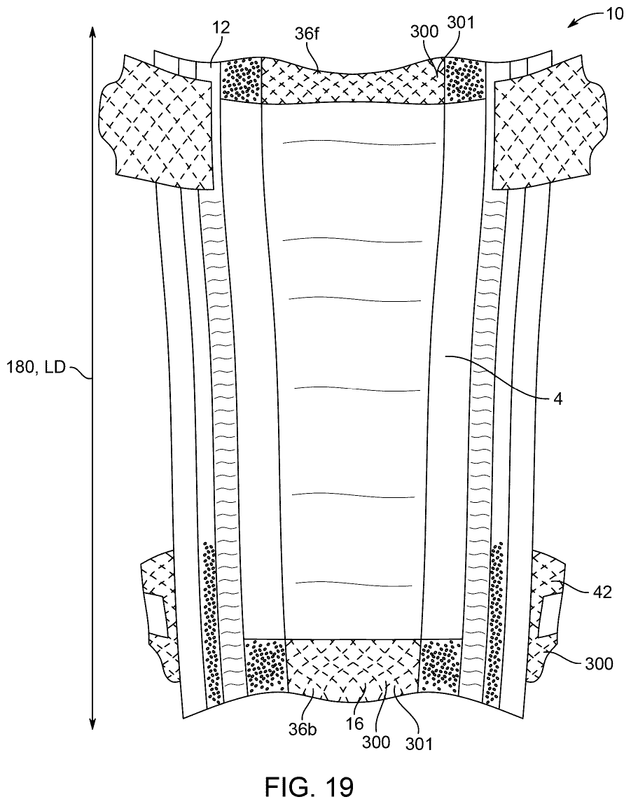

FIG. 18 is a perspective view of an absorbent article 10, laid flat, with a garment-facing surface 2 facing the viewer to show an exterior front waist region 12 comprising an ultrasonically bonded elastic laminate 300, 301 and an exterior back waist region 16 comprising an ultrasonically bonded elastic laminate 300, 301. The ultrasonically bonded elastic laminate 300, 301 may comprise a diamond pattern. FIG. 19 is a perspective view of an absorbent article 10, laid flat, with a wearer-facing surface 4 facing the viewer to show an interior front waist region 12 comprising an ultrasonically bonded laminate 300, 301 and an interior back waist region 16 comprising an ultrasonically bonded elastic laminate 300, 301. The ultrasonically bonded laminate 300, 301 may comprise a diamond pattern. The front waist region 12 may comprise a front elastic waistband 36f formed at least partially by the laminate 300, 301 and may have a front visual signal 202. The back waist region 16 may comprise a back elastic waistband 36b formed at least partially by the laminate 300, 301 and may have a back visual signal 204. The absorbent article 10 also may comprise back ears 42 comprising diamond pattern ultrasonically bonded elastic laminate 300, 301. The back ears 42 may be formed at least partially by the laminate 300, 301 and may comprise a visual signal 206 that corresponds with the front visual signal 202 and/or with the back visual signal 204.

Corresponding visual signals, for example, the front visual signal 202, the back visual signal 204, and/or visual signal 206, may be used to indicate that components of the diaper, for example, components (e.g., a front waistband 36f, a back waistband 36b, back ears 42, front ears 47, fasteners 46, a landing zone 44, an outer cover 40, a backsheet 28, and/or a topsheet 26) comprising the front visual signal 202, the back visual signal 204, and/or visual signal 206, should be aligned with each other when fastening the absorbent article 10. The implementation of corresponding visual signals, for example, may indicate which components to align with each other (for example, implementing corresponding visual signals on visually coordinated components (e.g., a front waistband 36f, a back waistband 36b, back ears 42, front ears 47, fasteners 46, a landing zone 44, an outer cover 40, a backsheet 28, and/or a topsheet 26) to indicate to a user to align the visually coordinated components) may result in an improvement in intuitiveness of fastening of the front and back of the absorbent article 10, improved placement of the absorbent article 10 on the wearer, and/or improved fit of the absorbent article 10 on the wearer.

In some configurations, the ultrasonically bonded elastic laminate 300 may be on the garment facing surface 2 of the absorbent article 10. In some configurations, the ultrasonically bonded elastic laminate 300 may be on the wearer facing surface 4 of the absorbent article 10. In some configurations, the ultrasonically bonded elastic laminate 300 may visible when viewing the garment facing surface 2 of the absorbent article 10. In some configurations, the ultrasonically bonded elastic laminate 300 may visible when viewing the wearer facing surface 4 of the absorbent article 10.

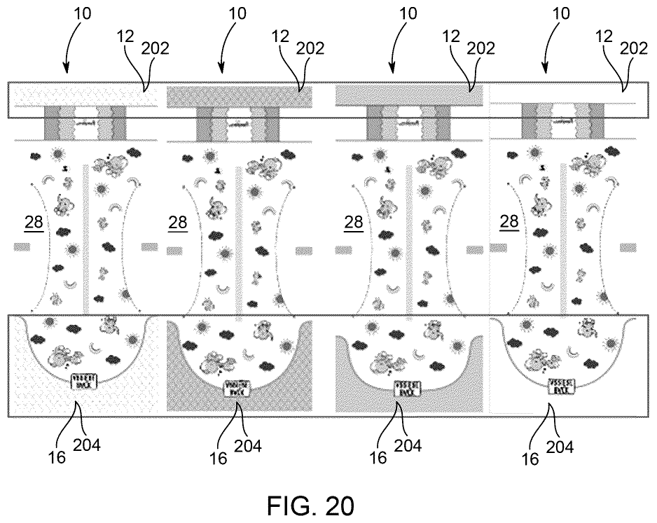

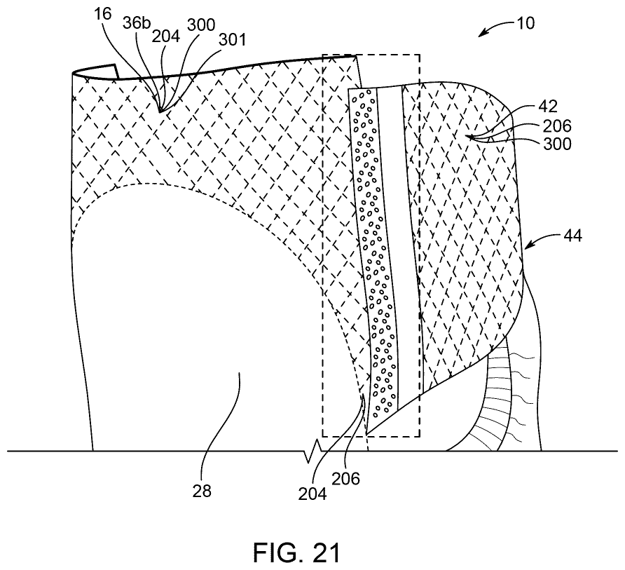

FIG. 20 is a plan view of several example absorbent articles 10 showing the front waist regions 12 and the back waist regions 16 of the back sheets 28. FIG. 21 is a perspective view of back ears 42 attached to a back sheet 28 fully within a back sheet visual signal area. The back ears 42 may be attached, for example, via bonds to back sheet 28 fully within the back sheet visual signal area.

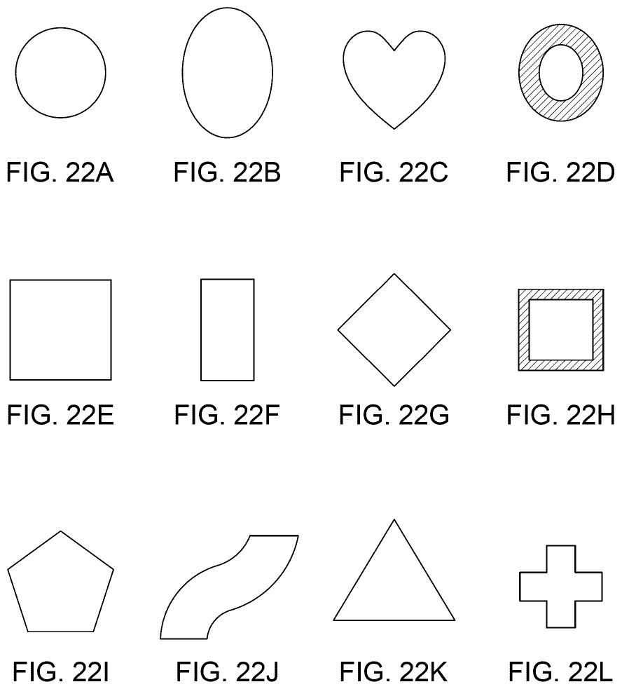

FIG. 22A-FIG. 22L are schematic illustrations of some exemplary ultrasonic bond patterns. As shown, the ultrasonic bonds may have a round shape, a curved shape, a shape with straight sides, or combinations thereof. It is to be appreciated that bonding may be present across the entire surface of any ultrasonic bond shape or may be present only at the perimeter. This distinction may be further appreciated by comparing FIG. 22E with FIG. 22H. Similar variations are contemplated for any given shape. It is also to be appreciated that the specific bond patterns shown are only examples and that any shape is possible. FIG. 22A shows an ultrasonic bond pattern having a circular shape. FIG. 22B shows an ultrasonic bond pattern having an oval shape. FIG. 22C shows an ultrasonic bond pattern having heart shape. FIG. 22D shows an ultrasonic bond pattern having ring shape with bonding only at the perimeter. FIG. 22E shows an ultrasonic bond pattern having square shape. FIG. 22F shows an ultrasonic bond pattern having rectangular shape. FIG. 22G shows an ultrasonic bond pattern having diamond shape. FIG. 22H shows an ultrasonic bond pattern having square shape with bonding only at the perimeter. FIG. 22I shows an ultrasonic bond pattern having n-side polygon shape. The specific n-sided polygon shown in FIG. 22I is a pentagon. It is to be appreciated that n may be any number greater than 3, such as for example, any number from 3 to 30. FIG. 22J shows an ultrasonic bond pattern having shape having both curved sides and straight sides. FIG. 22K shows an ultrasonic bond pattern having triangular shape. FIG. 22L shows an ultrasonic bond pattern having 12-sided shape in the form of a plus sign.

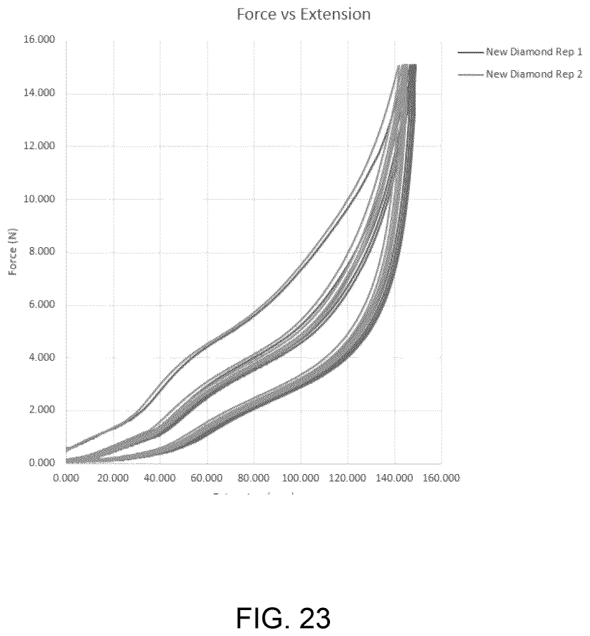

FIG. 23 is a chart plotting extension vs force curves for exemplary back ears.

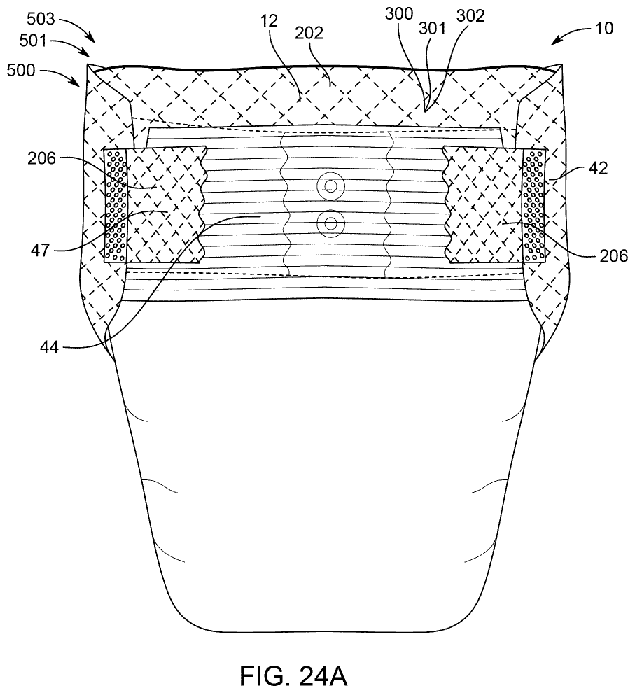

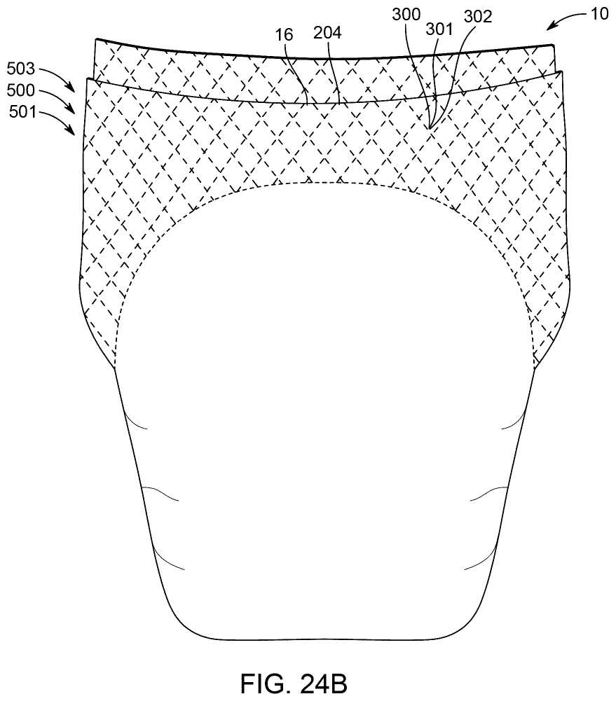

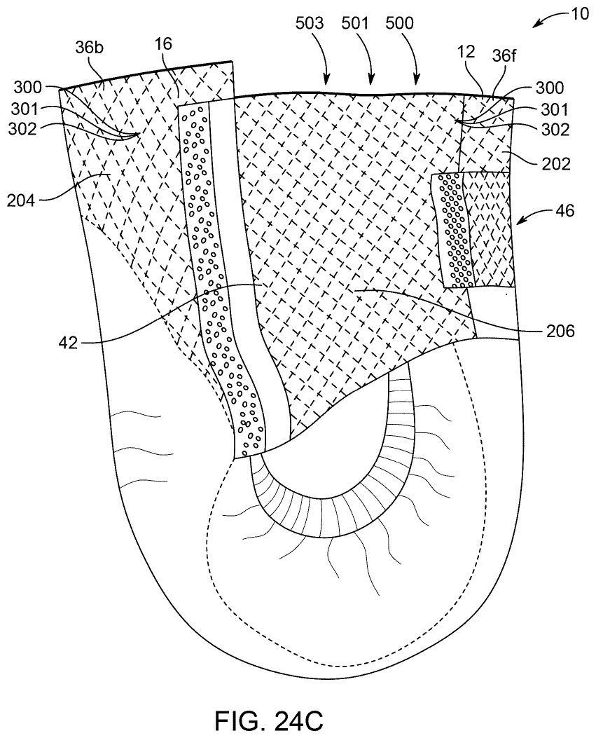

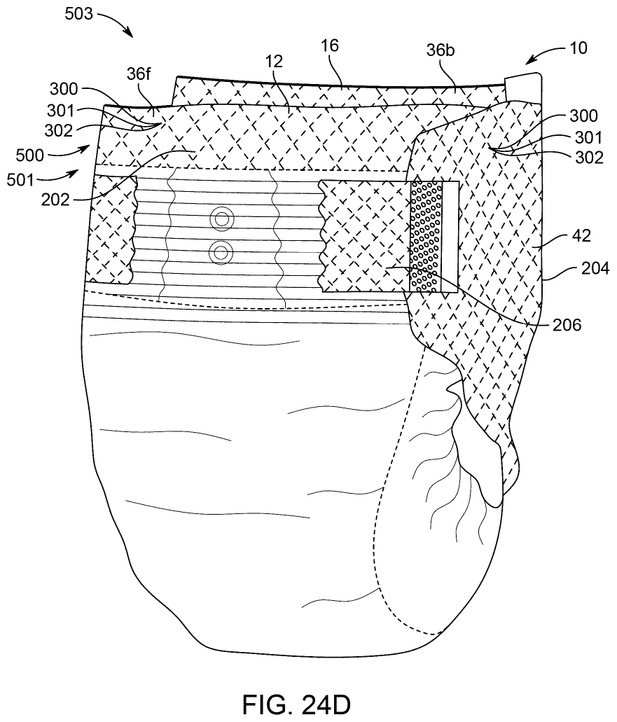

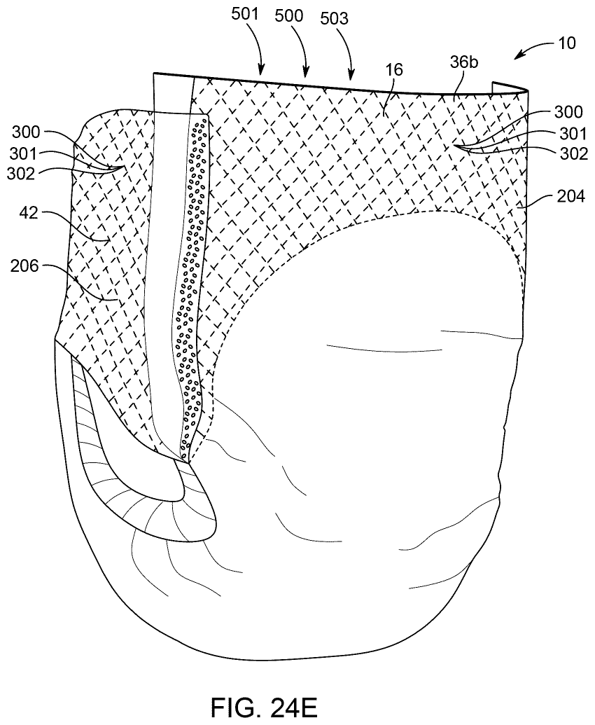

FIG. 24A is a front view of an absorbent article 10 comprising visually coordinated components 503. FIG. 24B is a back view of an absorbent article 10 comprising visually coordinated components 503. FIG. 24C is a side view of an absorbent article 10 comprising visually coordinated components 503. FIG. 24D is a front-side perspective view of an absorbent article 10 comprising visually coordinated components 503. FIG. 24E is a back-side perspective view of an absorbent article 10 comprising visually coordinated components 503.

### Integrated Waist Hoop

Referring to FIGS. 1-3 and 24A-24E, in some configurations, an absorbent article 10 may comprise an integrated waist hoop (IWH) 500 comprising a set of materials, features, and/or elements that form a continuous, or substantially continuous, visible circumference of the absorbent article 10 in the front waist region 12 and/or the back waist region 14. In some configurations, the IWH 500 may comprise an assembly of components in a continuous or substantially contiguous configuration encircling a wearer's waist, abdominal area, and/or a circumference of the absorbent article 10. In some configurations, the IWH 500 may cover about 40% to about 100% of the circumference of the absorbent article, about 60% to about 95% of the circumference of the absorbent article, or about 80% to about 90% of the circumference of the absorbent article, specifically reciting all 1% increments within the above recited ranges and all ranges formed therein or thereby. In some configurations, for example, the IWH 500 may comprise at least portions of a front waistband 36f, a back waistband 36b, back ears 42, front ears 47, fasteners 46, a landing zone 44, an outer cover 40, a backsheet 28, a topsheet 26, and/or bonded, seamed, and/or overlapped regions, for example bonded, seamed and/or overlapped regions forming a connection between the front waistband 36f, the back waistband 36b, the back ears 42, the front ears 47, the fasteners 46, the landing zone 44, the outer cover 40, the backsheet 28, and/or the topsheet 26.

In some configurations, the IWH 500 may comprise discontinuities along the circumference of the absorbent article 10. A discontinuity may have a length of about 20 mm or less, a length of about 10 mm or less, a length of about 5 mm or less, a length between about 1 mm and about 20 mm, or a length between about 0 mm and about 20 mm, specifically reciting all 1 mm increments within the above recited ranges and all ranges formed therein or thereby. In some configurations, the IWH may comprise five or fewer discontinuities along the circumference of absorbent article 10. In some configurations, the IWH 500 may comprise three or fewer discontinuities along the circumference of the absorbent article 10. In some configurations, the IWH 500 may comprise no discontinuities.

In some configurations The IWH 500 may comprise visually coordinated components, for example, as shown in FIGS. 24A-24E. The visually coordinated components may, for example, comprise visually coordinated signals, which may comprise one or more similar or substantially similar visual signals, for example a front visual signal 202, a back visual signal 204, and/or a visual signal 206. Such configurations may enhance the visual appeal of the multiple assembled components that make up the absorbent article 10. Although an absorbent article may still be perceived as comprising numerous assembled parts, one or more coordinated visual signals on certain components of the absorbent article, may render the article aesthetically pleasing as well as provide an intuitive application of the article to a wearer. For example, an absorbent article 10 comprising an IWH 500 comprising visually coordinated components comprising one or more coordinated visual signals may render the absorbent article aesthetically pleasing as well as provide an intuitive application of the article to a wearer. The one or more visual signals 202, 204, 206 may be strategically arranged and designed to convey a desirable and harmonious complex functionality without compromising softness and comfort of the absorbent article 10. For example, as shown in FIGS. 24A-24E, the absorbent article 10 may comprise an IWH 500 comprising a back waistband 36b at least a portion of a front waistband 36f, back ears 42, and fastening system components (e.g., fasteners 46), and the visual signals 202, 204, 206 may indicate to a user that these components should be aligned when coupling (e.g., applying) the fasteners to the landing zone 44. Indicating which components (e.g., a front waistband 36f, a back waistband 36b, back ears 42, front ears 47, fasteners 46, a landing zone 44, an outer cover 40, a backsheet 28, and/or a topsheet 26) should be aligned with each other may result in an improvement in intuitiveness of fastening of the front and back of the absorbent article 10, improved placement of the absorbent article on the wearer, and/or improved fit of the absorbent article 10 on the wearer.

In some configurations, the IWH 500 may comprise an internal integrated waist hoop (internal IWH) and/or an external integrated waist hoop (external IWH) 501. The internal IWH may comprise components visible when looking at the wearer-facing surface 4 of the absorbent article 10. The external IWH 501 may comprise components visible when viewing the garment-facing surfaces 2 of the absorbent article 10. The internal IWH may have a substantially identical, similar, complementary, or different appearance or pattern as compared to the external IWH 501.

In some configurations, the visual signals, for example, visual signals of the visually coordinated components of the IWH 500 may comprise a diamond pattern. In some configurations, a majority or all of the of the components of IWH 500 may comprise a diamond pattern. The geometry and/or dimensions of the diamond pattern may vary within each component or between some or all components of the IWH 500. For example, a diamond pattern in the waistband 36f 36b and a diamond pattern of the backsheet 28 may differ from each other, and the diamond pattern of the waistband 36f 36b and the diamond pattern of the backsheet may each differ from the diamond pattern on the front ear 47 or the back ear 42.

In some configurations, the diamond patterns may comprise a diamond shape. FIG. 26 depicts an example diamond shape. The diamond shape may comprise a first axis “a” and a second axis “b”. The first axis “a” and the second axis “b” may be measured between the two sets of opposing vertices, the first and second vertices, respectively. The first and second axes may be perpendicular to each other. The first vertices may be separated by first axis “a” and have a first angle of C, and the second vertices may be separated by second axis “b” and have a second angle D. The angles C plus D may equal 180 degrees or another angle.

In some configurations, a stretchable ear (e.g., front ear 47 or back ear 42) may comprise a diamond pattern formed by an ultrasonic bond pattern. When no strain is imparted to the material by the user, the first angle C may be between about 60 degrees and about 80 degrees, and the second angle D may be between about 120 degrees and about 100 degrees. In this form, the first axis “a” may have a length between about 3 mm and about 15 mm, between about 3 mm and about 13 mm, between about 5 mm and about 15 mm, or between about 5 mm and about 10 mm, specifically reciting all 1 mm increments within the above recited ranges and all ranges formed therein or thereby, and the second axis “b” may have a length between about 5 mm and about 20 mm, between about 10 mm and about 20 mm, between about 5 mm and about 15 mm, or between about 8 mm and about 13 mm, specifically reciting all 1 mm increments within the above recited ranges and all ranges formed therein or thereby, when the stretchable ear is in a relaxed condition. In this form, the area of the diamond pattern may be between about 10 mm² and about 80 mm², between about 20 mm² and about 70 mm², between about 30 mm² and about 60 mm², or between about 30 mm² and about 50 mm², specifically reciting all 1 mm² increments within the above recited ranges and all ranges formed therein or thereby.

In some configurations, when the stretchable ear is elongated under tension, the geometry of the diamond of the diamond pattern may change according to the amount of strain imparted to the ear by the user. For example, at full extension, the first angle C may be between about 95 and about 130 degrees, while the second angle D may be between about 85 degrees and about 50 degrees. In this form, the first axis “a” may have a length between about 5 mm and about 15 mm, between about 15 mm and about 25 mm or between about 10 mm and about 20 mm, specifically reciting all 1 mm increments within the above recited ranges and all ranges formed therein or thereby, and the second axis “b” may may have a length between about 5 mm and about 25 mm, between about 10 mm and about 25 mm, or between about 7 mm and about 15 mm, specifically reciting all 1 mm increments within the above recited ranges and all ranges formed therein or thereby, when the stretchable ear is fully extended. In this form, the area of the diamond pattern may be between about 65 mm² and about 95 mm², between about 50 mm² and about 110 mm², or between about 40 mm² and 100 mm², specifically reciting all 1 mm² increments within the above recited ranges and all ranges formed therein or thereby. For elongations intermediate the relaxed and fully extended states, the angles and axis lengths will have values intermediate the relaxed and fully extended values.

In some configurations, the diamond pattern may be formed on the backsheet 28 via a printing process. In this form, the first angle C may be between about 40 degrees and about 70 degrees and the second angle D may be between about 140 degrees and about 110 degrees. In this form, the first axis “a” may have a length between about 5 mm and about 20 mm, between about 10 mm and about 15 mm, or between about 15 mm and about 20 mm, specifically reciting all 1 mm increments within the above recited ranges and all ranges formed therein or thereby, and the second axis “b” may have a length between about 5 mm and about 20 mm, between about 10 mm and about 15 mm, or between about 7 mm and about 12 mm, specifically reciting all 1 mm increments within the above recited ranges and all ranges formed therein or thereby. In this form, the area of the diamond pattern may be between about 40 mm² and about 80 mm², between about 60 mm² and about 90 mm², or between about 50 mm² and about 70 mm², specifically reciting all 1 mm² increments within the above recited ranges and all ranges formed therein or thereby.

In some configurations, the first angle C may be between about 30 degrees and about 150 degrees, and the second angle D may be between about 150 degrees and 30 degrees. The first axis “a” may have a length between about 5 mm and about 30 mm and the second axis “b” may have a length between about 30 mm and about 5 mm. One of the axes of the diamond pattern may be parallel or substantially parallel to the longitudinal axis 50 of the absorbent article (e.g., within about 5 degrees) and the other may be generally parallel to the lateral axis 48 of the absorbent article 10 (e.g., within about 5 degrees). In some configurations, the first axis “a” may be parallel with the longitudinal axis 50 of the absorbent article 10. In some configurations, the first axis “a” may be disposed at any angle with respect to the longitudinal axis 180 of the absorbent article 10.

In some configurations, any visual signal, for example, front visual signal 202, back visual signal 204, and/or visual signal 206, and/or visually coordinated component of an IWH may comprise a diamond pattern and/or geometry that is identical, similar, or substantially similar to any diamond pattern and/or geometry described above. Any component described above comprising visual signals, including any visually coordinated component of an IWH may be elasticized and/or stretchable or not elasticized and/or not stretchable.

In some configurations, components in the front waist region 12 of the absorbent article 10 may comprise identical, similar, or substantially similar visual signals, for example, the front visual signal 202, the back visual signal 204, and/or the visual signal 206. In some configurations, only components in the front waist region 12 of the absorbent article 10 may comprise identical, similar, or substantially similar visual signals. In some configurations, only components in the rear waist region 16 of the absorbent article 10 may comprise identical, similar, or substantially similar visual signals. In some configurations, components in the front waist region 12 or the rear waist region 16 of the absorbent article 10 may or may not comprise identical, similar, or substantially similar visual signals.

Referring to FIGS. 1-2, 16-19, and 24A-24E, in some configurations, when the absorbent article 10 is in a laid-out state (e.g., no elastic contraction), visually coordinated components 503 (e.g., portions of an IWH 500) may overlap an axis 480 extending laterally between a first longitudinal end edge 481 of a back ear 42 of an absorbent article 10 and a second longitudinal end edge 482 of another back ear 42 of the absorbent article. In some configurations, the visually coordinated components 503 may overlap about 40% to about 100% of the axis 480, may overlap about 60% to about 90% of the axis 480, or may overlap about 80% to about 95% of the axis 480, specifically reciting all 1% increments within the above cited recited ranges and all ranges formed therein or thereby.

In some configurations when the absorbent article 10 is in a laid-out state (e.g., no elastic contraction), visually coordinated components 503 (e.g., portions of an IWH 500) may overlap of an axis 483 crossing laterally between a first longitudinal edge 484 of a front ear 47 of the absorbent article 10 and a second longitudinal end edge 485 of another front ear 47 of an absorbent article 10. In some configurations, the visually coordinated components 503 may overlap about 40% to about 100% of the axis 483, may overlap about 60% to about 90% of the axis 483, or may overlap about 80% to about 95% of the axis 483, specifically reciting all 1% increments within the above cited recited ranges and all ranges formed therein or thereby.

### Combinations

A1. An absorbent article comprising:
a liquid permeable topsheet;
a liquid impermeable backsheet;
an absorbent core positioned at least partially intermediate the topsheet and the backsheet; and
at least two discrete elasticized members;
wherein, each of the at least two discrete elasticized members comprises a front waistband, a back waistband, a pair of front ears, or a pair of back ears;
wherein the liquid impermeable backsheet comprises a front waist region and a back waist region separated from each other in a longitudinal direction;
wherein the liquid impermeable backsheet comprises a first visual signal in the front waist region and a second visual signal in the back waist region; and
wherein at least one of the discrete elasticized members comprises a third visual signal, wherein the third visual signal is the same as or substantially similar to the first visual signal and/or the second visual signal.

A2. The absorbent article of paragraph A1, wherein the first visual signal, the second visual signal, and the third visual signal comprise a color, a printed repeat pattern, a mechanically imparted repeat pattern or combinations thereof.

A3. The absorbent article of paragraph A1 or A2, wherein at least two of the discrete elasticized members comprise an ultrasonically bonded laminate.

A4. The absorbent article of paragraph A1 or A2, wherein all of the discrete elasticized members comprise an ultrasonically bonded laminate.

A5. The absorbent article of any of paragraphs A1-A4, wherein at least two of the discrete elasticized members have a minimum extension of 5 mm at a load of 400 g or a minimum extension of 22 mm at a load of 1000 g.

A6. The absorbent article of any of paragraphs A1-A4, wherein at least all of the discrete elasticized members have a minimum extension of 5 mm at a load of 400 g or a minimum extension of 22 mm at a load of 1000 g.

A7. The absorbent article of paragraph A2, wherein the color comprises grey, mint green, yellow, teal, pink, red, orange or white.

A8. The absorbent article of paragraph A2, wherein the printed repeat pattern or mechanically imparted repeat pattern comprises a parallelogram, a linear array of shapes, or an offset array of shapes.

A9. The absorbent article of paragraph A2, wherein the printed repeat pattern or the mechanically-imparted repeat pattern remain substantially the same when the absorbent article is relaxed or stretched.

A10. The absorbent article of paragraph A3, wherein the laminate comprises a preactivated film, skins with wrinkles and furrows or visible lines of preactivation.

A11. The absorbent article of any of the preceding paragraphs A1-A10, wherein the absorbent article comprises a taped diaper.

A12. The absorbent article of paragraph A1, wherein the absorbent article comprises an integrated waist hoop comprising the at least two discrete elasticized members wherein all of the at least two discrete elasticized members comprise a third visual signal, and wherein the third visual signal is the same as or substantially similar to the first visual signal and/or the second visual signal.

A13. The absorbent article of paragraph A12, wherein the integrated waist hoop has five or fewer discontinuities.

A14. The absorbent article of paragraph A12, wherein portions of the integrated waist hoop comprising the third visual signal are configured to overlap at least 60% of an axis laterally extending between a first back ear longitudinal edge and a second back ear longitudinal edge when the absorbent article is in flat, non-contracted, laid-out state.

A15. The absorbent article of paragraph A12, wherein portions of the integrated waist hoop comprising the third visual signal are configured to overlap at least 60% of an axis extending laterally between a first front ear longitudinal edge and a second front ear longitudinal edge when the absorbent article is in a flat, non-contracted, laid-out state.

A16. A disposable absorbent article comprising:
a liquid permeable topsheet;
a liquid impermeable backsheet;
an absorbent core positioned at least partially intermediate the topsheet and the backsheet; and
at least two discrete elasticized members; and
a front waist region and a back waist region separated from each other in a longitudinal direction;
wherein, each of the at least two discrete elasticized members comprises a front waistband, a back waistband, a pair of front ears, or a pair of back ears;
wherein a first discrete elasticized member of the at least two discrete elasticized members comprises a first visual signal;
wherein a second discrete elasticized member of the at least two discrete elasticized members comprises a second visual signal;
wherein the second visual signal is the same as or substantially similar to the first visual signal; and
wherein both the first visual signal and the second visual signal are at least partially in the front waist region or the both the first visual signal and the second visual signal are at least partially in the back waist region.

A17. The absorbent article of paragraph A16, wherein both the first visual signal and the second visual signal are at least partially in the front waist region;

A18. The absorbent article of paragraph A16, wherein the first visual signal comprises a color and a mechanically imparted pattern.

A19. The absorbent article of paragraph A16, wherein the first visual signal comprises a color and a printed pattern.

A20. A disposable absorbent article comprising:
a liquid permeable topsheet;
a liquid impermeable backsheet;
an absorbent core positioned at least partially intermediate the topsheet and the backsheet; and
at least two discrete elasticized members;
a front waist region and a back waist region separated from each other in a longitudinal direction; and
an integrated waist hoop comprising the at least two discrete elasticized members;
wherein the at least two discrete elasticized members are at least partially in the front waist region and/or the back waist region;
wherein a first discrete elasticized member of the at least two discrete elasticized members comprises a first visual signal and a second discrete elasticized member of the at least two discrete elasticized members comprises a second visual signal, wherein the second visual signal is the same as or substantially similar to the first visual signal; and
wherein the waist hoop covers at least 80% of a circumference of the absorbent article.

B1. An absorbent article comprising:
a liquid permeable topsheet;
a liquid impermeable backsheet;
an absorbent core positioned at least partially intermediate the topsheet and the backsheet; and
at least two visually coordinated components;
a front waist region and a back waist region separated from each other in a longitudinal direction; and
an integrated waist hoop comprising the at least two visually coordinated components;
wherein the at least two visually coordinated components are at least partially in the front waist region and/or the back waist region;
wherein the at least two visually coordinated components comprise a front waistband, a back waistband, one or more back ears, one or more front ears, one or more fasteners 46, one or more landing zones, an outer cover 40, the backsheet 28, and/or a topsheet 26.

B2. The absorbent article of paragraph B1, wherein the visually coordinated components each have the same, similar, or substantially similar visual signal.

B3. The absorbent article of paragraph B2, wherein the visual signal comprises color, a mechanically imparted pattern, and/or a printed pattern.

B4. The absorbent article of any of paragraphs B1-B3, wherein the integrated waist hoop comprises 3 or fewer discontinuities.

B5. The absorbent article of any of paragraphs B1-B4, wherein the integrated waist hoop covers 65-95% of the circumference of the absorbent article.

B6. The absorbent article of any of the paragraphs B1-B4, wherein the integrated waist hoop covers 80-95% of the circumference of the absorbent article.

B7. The absorbent article of any of the paragraphs B1-B4, wherein the integrated waist hoop covers 40-65% of the circumference of the absorbent article.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A disposable absorbent article comprising:
a liquid permeable topsheet;
a liquid impermeable backsheet;
an absorbent core positioned at least partially intermediate the topsheet and the backsheet; and
at least two discrete elasticized members;
wherein, each of the at least two discrete elasticized members comprises a front waistband, a back waistband, a pair of front ears, or a pair of back ears;
wherein the liquid impermeable backsheet comprises a front waist region and a back waist region separated from each other in a longitudinal direction;
wherein the liquid impermeable backsheet comprises a first visual signal in the front waist region and a second visual signal in the back waist region; and
wherein at least one of the discrete elasticized members comprises a third visual signal, wherein the third visual signal is the same as or substantially similar to the first visual signal and/or the second visual signal.

2. The absorbent article of claim 1, wherein the first visual signal, the second visual signal, and the third visual signal comprise a color, a printed repeat pattern, a mechanically imparted repeat pattern or combinations thereof.

3. The absorbent article of claim 1, wherein at least two of the discrete elasticized members comprise an ultrasonically bonded laminate.

4. The absorbent article of claim 1, wherein all of the discrete elasticized members comprise an ultrasonically bonded laminate.

5. The absorbent article of claim 1, wherein at least two of the discrete elasticized members have a minimum extension of 5 mm at a load of 400 g or a minimum extension of 22 mm at a load of 1000 g.

6. The absorbent article of claim 1, wherein at least all of the discrete elasticized members have a minimum extension of 5 mm at a load of 400 g or a minimum extension of 22 mm at a load of 1000 g.

7. The absorbent article of claim 2, wherein the color comprises grey, mint green, yellow, teal, pink, red, orange or white.

8. The absorbent article of claim 2, wherein the printed repeat pattern or mechanically imparted repeat pattern comprises a parallelogram, a linear array of shapes, or an offset array of shapes.

9. The absorbent article of claim 2, wherein the printed repeat pattern or the mechanically imparted repeat pattern remain substantially the same when the absorbent article is relaxed or stretched.

10. The absorbent article of claim 3, wherein the laminate comprises a preactivated film, skins with wrinkles and furrows or visible lines of preactivation.

11. The absorbent article of claim 1, wherein the absorbent article comprises a taped diaper.

12. The absorbent article of claim 1, wherein the absorbent article comprises an integrated waist hoop comprising the at least two discrete elasticized members, wherein all of the at least two discrete elasticized members comprise a third visual signal, and wherein the third visual signal is the same as or substantially similar to the first visual signal and/or the second visual signal.

13. The absorbent article of claim 12, wherein the integrated waist hoop has five or fewer discontinuities.

14. The absorbent article of claim 12, wherein portions of the integrated waist hoop comprising the third visual signal are configured to overlap at least 60% of an axis laterally extending between a first back ear longitudinal edge and a second back ear longitudinal edge when the absorbent article is in a flat, non-contracted, laid-out state.

15. The absorbent article of claim 12, wherein portions of the integrated waist hoop comprising the third visual signal are configured to overlap at least 60% of an axis extending laterally between a first front ear longitudinal edge and a second front ear longitudinal edge when the absorbent article is in a flat, non-contracted, laid-out state.

16. A disposable absorbent article comprising:
a liquid permeable topsheet;
a liquid impermeable backsheet;
an absorbent core positioned at least partially intermediate the topsheet and the backsheet; and
at least two discrete elasticized members; and
a front waist region and a back waist region separated from each other in a longitudinal direction;
wherein, each of the at least two discrete elasticized members comprises a front waistband, a back waistband, a pair of front ears, or a pair of back ears;
wherein a first discrete elasticized member of the at least two discrete elasticized members comprises a first visual signal;
wherein a second discrete elasticized member of the at least two discrete elasticized members comprises a second visual signal;
wherein the second visual signal is the same as or substantially similar to the first visual signal; and
wherein both the first visual signal and the second visual signal are at least partially in the front waist region or the both the first visual signal and the second visual signal are at least partially in the back waist region.

17. The absorbent article of claim 16, wherein both the first visual signal and the second visual signal are at least partially in the front waist region.

18. The absorbent article of claim 16, wherein the first visual signal comprises a color and a mechanically imparted pattern.

19. The absorbent article of claim 16, wherein the first visual signal comprises a color and a printed pattern.

20. A disposable absorbent article comprising:
a liquid permeable topsheet;
a liquid impermeable backsheet;
an absorbent core positioned at least partially intermediate the topsheet and the backsheet; and
at least two discrete elasticized members;
a front waist region and a back waist region separated from each other in a longitudinal direction; and
an integrated waist hoop comprising the at least two discrete elasticized members;
wherein the at least two discrete elasticized members are at least partially in the front waist region and/or the back waist region;
wherein a first discrete elasticized member of the at least two discrete elasticized members comprises a first visual signal and a second discrete elasticized member of the at least two discrete elasticized members comprises a second visual signal, wherein the second visual signal is the same as or substantially similar to the first visual signal; and
wherein the integrated waist hoop covers at least 80% of a circumference of the absorbent article.
